(19) United States
(12) Patent Application Publication
(10) Pub. No.: US 2005/0142075 A1
(43) Pub. Date: Jun. 30, 2005

(54) Breath equalizing mint

(76) Inventor: Rodger H. Rast, Gold River, CA (US)

Correspondence Address: RODGER H. RAST, 11230 GOLD EXPRESS DRIVE, SUIT 310 MS 337, GOLD RIVER, CA, 95670, US

(21) Appl. No.: 11/060,108

(22) Filed: Feb. 16, 2005

Related U.S. Application Data
(63) Continuation-in-part of application No. 10/016,852, filed on Dec. 13, 2001.

(51) Int. Cl. A61K 9/68; A61K 7/16

(52) U.S. Cl. 424/48; 424/49

(57) ABSTRACT

A breath “mint” is described which reduces the offense particularly for those who have eaten onions or garlic, or other specific odiferous elements. The invention may be packaged as a mint or any other form of breath application (e.g., strips, drops, capsules, and so forth). The “mint” is configured to equalize the breath of the partakers. The party without the offensive breath takes the “mint”, which contains elements to reduce the partakers sensitivity to the breath condition of the other party. According to a preferred embodiment both the offender and offendee take the mints. In one embodiment the mints taken by the offendor and offendee differ, with the one taken by the offendee containing the equalizing additions. The mint system is especially beneficial in close romantic situations, where the partial odor masking of traditional mints is insufficient.

### CROSS-REFERENCE TO RELATED APPLICATIONS

This application is a continuation-in-part and claims priority from patent application Ser. No. 10/016,852 filed Dec. 13, 2001, issued at U.S. Pat. No. ______. This application claims also priority from U.S. provisional application Ser. No. 60/256,887 filed on Dec. 15, 2000 and Ser. No. 60/256/888 filed on Dec. 15, 2000 which are incorporated herein by reference.

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

1. Field of the Invention

The invention pertains generally to consumables and more particularly to a breath mint, or breath preparation for certain forms of oral halitosis.

2. Description of the Background Art

The number of products in the consumables category continues to rise, and the need for continuous improvement provides a competitive edge for manufacturers.

Consumers are often very concerned with breath halitosis and each year they consume a variety of breath preparations, such as so-called “breath mints” and various concoctions in order to eliminate the odor so as not to offend other nearby persons. Estimates for 2002 indicated over the counter sales of breath preparations in the United States was about $3.7 Billion. The breath mint market is growing readily: during 2004 the breath freshener category was up 15.3 percent, while the rest of the candy market grew only 3.2 percent.

Most often these breath mints are consumed by a person that has eaten an odiferous item (such as garlic/onions, fish, hot spices), or smoked a cigarette, in an attempt to mask, or preferably reduce, the odors so that a spouse or significant other will not be put-off by the smell of their breath. The best that the majority of breath fresheners can hope to achieve it that of masking the odor by mixing in a strong spearmint, peppermint, wintergreen, spice (i.e. cinnamon), or fruity flavor to cover the unpleasant odor. Some breath applications also use parsley or other spices such as Cardamom or similar in attempting to combat the problem. The use of breath mints is thereby generally an incomplete remedy to such strongly pungent forms of breath born halitosis.

The portion of the odor associated with common breath related halitosis is considered to result from the activity of anaerobic bacteria within the mouth and specifically at the back of the tongue which generate volatile sulfur compounds as a byproduct of breaking down sulfur-containing amino acids, and elements of specific food groups. Sulphides comprise a number of the identified breath odiferants, which stem from a number of sources. However, not all of the odor associated with these items originates from the mouth, as it has been found that after consumption of certain foods, such as garlic and onions, the food is absorbed into the bloodstream and transferred to the lungs wherefrom the odiferant may be expelled with the breath. Breath fresheners typically are packaged as drops, chewing gums, or in various tablet, lozenge, and gel forms. Most common is the mint form of breath freshener which is generally manufactured from a sugar, or sugar substitute, in addition to other ingredients including coloring and flavoring agents, such as mint-based or spice-based flavors.

Unfortunately the masking of an odor such as that of garlic, or onions, such as with a minty flavoring results in a strange odor of minty garlic. The resulting odd mixture of odors is often still strongly offensive, and the unusual combination may perhaps be likened to the mixing of baking chocolate within a tuna casserole, . . . a truly unpleasant combination.

Therefore, a method and consumable tablet, lozenge, capsule, gel-strip, gum, liquid, or equivalent, are needed that will reduce the offensive nature of the odor to allow for comfortably closeness to be achieved between spouses or significant others.

The use of manufactured firelogs has increased as consumers more often seek the warmth and comfort of a fireplace fire without the bother of starting a wood fire that requires the use of kindling, and other fire starting materials. In addition, the manufactured firelogs are typically safer and require less “tending” than wood fires. Furthermore, recent advances in firelog materials have resulted in the production of firelogs which burn cleaner than those of wood burning fires.

One of the determining factors for a consumer choosing a manufactured firelog, is the duration of the fire that it will produce. It will be appreciated that an artificial firelog, once consumed in flames, may not be readily, or advisably, extinguished. Extinguishing a lit firelog is a risky proposition as the flammable waxes can be easily spread to overheat the fireplace or consume materials outside the fireplace area.

In addition, the consumer has no control over the heat generated by the use of the manufactured firelog. These logs will generate a given amount of heat for their specific size and configuration in relation to the draft through the fireplace and the air-space existing about the log. Often the burning of a manufactured firelog product can generate such a large quantity of heat that those sitting in front of the fireplace can be made uncomfortable, however, they have had no recourse. Furthermore, flue fires can result when a fire produces excessive heat for the given fireplace, or when the flue has not been regularly maintained. This is especially true today as a large percentage of all fireplaces made today are small drop-in units that employ small cylindrical metal flues. In many cases fireplaces are never, or rarely, cleaned, such is the case with a number of apartment complexes. Houses and apartments have been burnt to the ground in numerous instances in which flue residues catch fire at a high heat intensity and therein ignite nearby structural elements of the dwelling. Further complicating this is the nature of the construction on these logs. A conventional fire can be prodded and arranged to reduce the size of the flames and thereby reduce the risk. A manufactured firelog, by contrast, will generate MORE heat if disturbed as it breaks up into tiny fragments so that more of the wood pulp impregnated with flammable residues are available to burn. Currently, consumer choices are limited with regard to the choice of manufactured firelog products.

As can be seen therefore a need exists for a firelog, and method of manufacturing same, that allows consumers to control the burn rate and heat production of a manufactured firelog product prior to ignition of that product. The consumption rate adjustable firelog products in accordance with the present invention satisfies those needs, as well as others, and overcome deficiencies in previously known techniques.

Traditionally, buns and other similar forms of baked goods are configured for receiving food items (fillings and condiments) between two edge-joined halves-portions of bread forming the bun. Typically, the dough for the buns is baked and then partially cut-through so that it can be opened to allow for the insertion of various ingredients and condiments therein. Perhaps the prime example of these baked goods are the buns used for hot dogs. Generally cylindrical in shape, the hot dog bun is cut lengthwise so that it may be opened for receiving a wiener. The depth of the cut can extend beyond 90% of the width of the roll to allow the bun to be opened while still providing an enclosed “V” or “U” shaped structure.

In preparing a “hot dog”, for example, the consumer gently opens the bun, prepares it with condiments, inserts the wiener, and then continues to add additional desired ingredients such as relish and so forth. It is very beneficial that the halves of the bun stay joined so that the hot dog and various, often messy, condiments are retained between the halves of the bun. Unfortunately split buns, such as hot dog buns, often separate at the hinge either upon opening, or after being loaded with often wet, or moist, ingredients, whereupon the consumer must fumble, in generally futile attempts, to keep the various ingredients from departing by way of the rear of the bun. Consumers utilizing other forms of baked goods, such as hoagie rolls, sandwich rolls, hamburger buns, and the like, often experience similar “separation” anxieties. The problem of bun separation is so pervasive that one large chain of sandwich shops specializing in subs, or submarine sandwiches, has established a portion of their reputation on their method of cutting the bun by removing a top v-shaped portion, wherein the elevated sides of the roll can still retain the ingredients. Although the method suffers from a number of shortcomings, such as not being able to get ingredients to cover the full span of the roll, it may be an improvement for use with submarine sandwiches.

As can be seen, therefore, the development of a bun and method of preventing bun separation for use with various baked goods would be of benefit to hot dog consumers as well as those whose delight can be found in submarine sandwiches, hamburgers, hoagies, steak sandwiches, poor-boy sandwiches, and other foods which utilize forms of split roll baked goods. The split resistant buns and rolls according to the present invention satisfies those needs, as well as others, and overcome deficiencies in previously known techniques.

Traditionally wine bottles have been stoppled using a cylindrical section of cork that is press-fit into the bottle opening. Conventional cork removal 200 is depicted in FIG. 23, wherein a bottle 202 has an inserted cork 204 which is being removed by corkscrew 206 which has a threaded end 208 and handle 210, which removes cork 204 from the neck 212 of the bottle. It will be appreciated that the consumer must supply the cork removal device in order to slidable remove the cork from the bottle.

The cork not only stopples the bottle but it imparts specific flavoring to the wine while during aging. A wax is applied to the cylindrical exterior of the corks and the corks are inserted into a filled bottle under a substantial force. The removal of a cork from the bottle also requires the application of a significant removal force, such as between 20-50 pounds. Various cork removal devices have been generally utilized, such as cork removers that utilize screw threads, tines, and the application of gaseous pressure. However, in order to enjoy the libation, the consumer is required to find a cork removal device.

Packaging a cork removal device with the wine bottle has been proposed, however, the cost of these devices is often prohibitive for general use. Currently, these integrated cork removal devices utilize a threaded-shank, of non-reactive material, whose top protrudes from the top of the bottle. The threading of a permanent shank within the cork can damage the cork and subjects the cork material to sustained pressures. In addition, the user can not utilize most forms of conventional cork removal devices due to the central presence of the permanent shank. The user must utilize a specialty cork removal device designed to remove the specific cork, which hopefully has not been misplaced, or been broken.

Therefore, it will be appreciated that the current methods of corkage and cork removal do not economically allow packaging of a cork remover with the bottle while allowing consumer to remove the cork with their conventional cork-removal devices. The present invention allows a cork to be removed by a method chosen at the discretion of the consumer, and can be configured so that external removal devices are not required.

Bathroom mirrors are subject to steam from showers which can condense on mirror surfaces to “fog them up” thus making them unusable by the consumer when grooming and applying cosmetics. Traditionally, a consumer that needs the use of the fogged-up mirror without waiting for it to unfog, has had to attempt to wipe the surface of the mirror with a nearby towel, or perform other time consuming and often unsatisfactory means of unfogging a portion of the surface of the mirror. It will be appreciated by anyone relying on such techniques that they are time consuming and generally do an inadequate job of removing the fog without dirtying the surface of the mirror.

Therefore, a need exists for a method and apparatus for easily removing the fog from mirror surfaces, such as in bathrooms, which are exposed to steam.

### BRIEF SUMMARY OF THE INVENTION

Improvements to a number of consumables is described that facilitate use by the consumer.

The present invention includes a breath preparation that eliminates the offensive nature of particular classes of consumed materials, such as onions and garlic, which is generally referred to herein as a breath “equalizer”. Traditional breath mints and similar preparations are directed at masking the offensive odor with strong flavors, such as mints and spices. However, the odor of garlic or onions, and other strong odiferous items are difficult to completely overcome, and current breath preparations are ineffective at completely overcoming the offensive odor.

The present invention takes a different direction than current breath preparations. It has been recognized within the present invention that the real problem with having breath tainted by a particular odiferous substance, such as garlic and onions, or smoking, arises when a given party “A” which has consumed a pungent article, (or smoked), desires close contact with another party “B”. If party “A” were isolated, there would be no issue with having onion, or garlic breath; it wouldn't matter as party “A” is not offended by their own breath—they are acclimated to it and don't even notice they have odiferous breath. The issue with the odiferous breath of party “A's” “aromatic breath” arises when they attempt to come in close contact with a party “B” such as a spouse, or significant other, that has not similarly partaken of the odiferous element. In many cases party “B” having not partaken in the same pungent consumption can be aromatically offended by the odor which restrains their desire for close contact with party “A”. It will be appreciated that even the ingestion of a small quantity of garlic, or onions, may taint one's breath for up to about three days. Conventional breath mints are directed for consumption by party “A” to reduce the offense that their pungent breath has on party “B”.

The present invention further recognizes persons are not sensitive to their own breath as they are acclimated to it. The olfactory senses become desensitized to an aroma that is ever-present. The present invention is based on the concept that it is far easier to “counterbalance”, or “equalize” a pungent aroma than to cover it up. Therefore, the present invention describes a breath preparation which incorporates specific odiferous agents, referred to herein as odiferants, for consumption by the offended party “B”, whereupon they will become readily acclimated to the aromatic elements and no longer be offended by the breath of party “A”. For example, a breath “mint”, or perhaps more accurately referred to as a “breath equalizer”, is created containing traces of sulfide compounds (i.e., forms of mercaptans) and other associated odiferants and intermediary compounds that mirror the scent of those found as a result from eating garlic and onions, or any other particular class of pungent aromatic element. The breath equalizer is to be consumed by the person whose olfactory senses are offended (Party “B”), instead of by the person whose breath is odiferous (Party “A”). It will be appreciated that after orally consuming even a very small amount of the odiferous compounds within the breath equalizer, that party “B” will no longer be offended by the breath of party “A”—wherein they are equalized with regard to the particular element being sensed, such as a mercaptan within onions.

It will be appreciated that consumers may be timid about taking a breath preparation that is touted as a breath equalizer, or as containing an odiferant, whereby the breath preparation of the present invention may be otherwise described as reducing “scent sensitivity” for the offended individual to one or more specific target items, such as garlic and onions. It will also be appreciated that the odiferant preferably does not constitute the taste of the item being mimicked, such as garlic or onion, only the nature and concentration of this background aroma.

The breath equalizers according to the invention may be created for various types of breath situations, such as garlic and onion breath, smokers breath, and so forth. The present invention may be manufactured in any conventional breath preparation form, such as hard pill-form lozenges, gelcaps (and similar), chewing gum, drops, and so forth without departing from the teachings of the present inventions. These breath equalizer are directed for use by the offended party instead of the offending party.

In addition, the present invention describes the use of the breath equalizing agents in combination with traditional breath fresheners. The mint, or other traditional odor hiding aromatic flavor, is combined with the equalizing ingredients for use by the offended party “B”, or both parties “A” and “B”.

Furthermore, an embodiment is described wherein a package contains two forms of breath preparations. A first breath preparation is configured as a traditional breath freshener for consumption by party “A” (that partook in eating garlic, smoking, or other specific pungent activity). A second breath preparation intended for consumption by party “B”, incorporates specific odiferous compounds to equalize the breath odor of the other party. Preferably, both of the preparations utilize the same base, such as a traditional mint or other freshener, while the second breath preparation will additionally contain the odiferant. The use of a common breath freshening base within this latter form of specifically directed breath preparation, provides a common flavoring which further aids in reducing the detectably breath differences between party “A” and party “B”.

By way of example, an embodiment is described wherein a package of lozenge style breath preparations, commonly referred to as breath mints, is adapted with a certain quantity of specially marked “mints” which are directed for use by the other party as breath equalizers. These may be “marked” by color, shape, indicia, and so forth, or by packaging them separately, such as within a opposing end of a mint roll, a separate compartment, and other ways of indicating the different object of the mints. It will be appreciated, therefore, that the present invention provides a breath preparation which may be utilized effectively by an offended party, by both parties, or wherein separate breath preparations for each of the two parties is created which reduces offensive effects. The breath equalizers of the present invention are manufactured in the form of a comestible, such as tablets, pills, lozenges, chewing gums, powders, mouth strips, along with liquid form breath preparations.

The present invention is amenable to embodiment in a number of different forms according to the teachings found herein. One such embodiment describes a breath equalizer preparation, comprising: (a) an edible base material; (b) flavorings within the edible base material to enhance flavor; and (c) at least one odiferant agent within the edible base material which generates or simulates one or more specific causes of breath-born halitosis; and (d) wherein the consumption of the odiferant agent within the base material by a consumer causes a reduction in the olfactory sensitivity of the specific causes of the breath-born halitosis associated with the odiferant.

The odiferant can comprise any compounds which are directly sensed as the odiferant, or similate the effect of that odiferant, or that act as intermediaries with other compounds to create the odiferant. One such class of compounds are select sulphides and precursors associated with a given breath-born form of halitosis. In one embodiment of the invention the particular odiferant is associated with the consumption of garlic, or onions. Wherein ingesting the equalizing breath preparations has a similar effect, in relation to the ability to sense the smell of onions and garlic, as eating a small quanity of onions or garlic. For example, the odiferant may include quantities of acrylonitrile, allicin sulphide, or a combination thereof, to decrease olfactory sensitivity to garlic and onions. The base material of the breath preparation preferably includes breath freshening ingredients, in particular when used in combination with a conventional breath freshening mint used for the offending party, such as has eaten onions and/or garlic. The breath equalizer preparation can be created in any convention form, however, it is preferably selected from breath preparation carriers as selected from the group of carriers for oral consumption consisting of tablets, lozenges, chewing gums, gel strips and liquid preparations.

In a preferred embodiment the breath equalizer preparation is packaged in combination with conventional breath preparations. The portions of the package, or preparations contained therein, are marked to designate their respective use as breath freshener, or breath unfreshener. In use, a first consumer having a particular halitosis (i.e., garlic/onion) can take a conventional breath mint from the package and offer a second consumer, not having that particular form of breath halitosis, a breath unmint from the same package. The breath of the second individual is then equalized by the odiferant, wherein their sensitivity to that particular form of halitosis present in the breath of the first consumer is lowered.

The present invention can also be described as a method of creating a breath equalizer preparation which reduces the sensitivity of the person consuming the preparation to specific elements of breath-born halitosis, comprising: (a) formulating an edible base material; (b) adding specific odiferants to the base material which are associated with specific forms of breath-born halitosis; and (c) packaging the base material into a consumable form. In a preferred embodiment the breath equalizer preparation is sold alongside, or more preferably packaged in combination with, conventional breath fresheners. When sold in combination with more conventional breath preparations, (without the odifericant) the mints (e.g., tablets, lozenges, strips, liquids, and other package forms) are preferably marked so that they may be discerned from conventional breath preparations which are directed only at attempting to freshen the breath.

An object of the invention is to reduce the offense taken by a spouse or other significant party “B” when a party “A” is subject to particular breath-born odor.

Another object of the present invention is to provide a breath preparation which is directed for use by the party which is offended, as opposed to the party that is causing the odor offense.

Another object of the present invention is to provide a breath preparation which does not rely on attempts at masking strong odors such as garlic/onions, or smoking.

Another object of the present invention is to provide a breath preparation system which is configured for overcoming the offense taken by specific maloderants.

Another object of the present invention is to provide a breath preparation that may be manufactured in various flavors, forms, and types.

Another object of the present invention is to provide a breath preparation which is provides a two part solution to offensive mouth odors, one for the party causing the offense and a second part for the offended party.

Another object of the present invention is to provide a breath preparation that may be easily manufactured at low cost and using substantially conventional processes.

Further objects and advantages of the invention will be brought out in the following portions of the specification, wherein the detailed description is for the purpose of fully disclosing preferred embodiments of the invention without placing limitations thereon.

The present invention describes a manufactured firelog and method of making firelog products, in which the consumption rate of the firelog may be set by the consumer prior to starting the fire. The benefits of providing an adjustable consumption rate will be readily appreciated, in that the consumer will no longer be required to purchase logs with different burning intervals, or be subjected to firelogs generating excessive heat levels. In addition firelog products are described which make it easier for the user to select the desired intensity and duration of a fire at the time of use, instead of being required to decide at the time the firelog product is purchased.

Manufactured firelogs are manufactured by agglomerating combustible materials, such as wood chips and wood containing by-products with binding agents and combustion aids, such as wax. Typically, manufactured firelogs are produced within an extrusion process. It will be recognized that when burning a log, the maximum heat output is generated when the largest amount of log surface area is being combusted in the flames while the oxygen within the surrounding air freely circulates or is being forced into the fire surrounding the firelog. The amount of heat generated by the firelog is a function of the surface area consumed by flame.

The present invention recognizes that the problem faced by consumers with regard to manufactured firelog size selection, and excessive heat generation are both related to the consumable surface area of the firelog product which is generally manufactured by agglomerating a combustible material into a shape such as a firelog shape. One aspect of the present invention are firelogs manufactured with an integral combustion shield for covering a portion of the surface area of the firelog that may be selectively utilized to set the duration and burning rate as the manufactured firelog is consumed during the fire.

By way of example and not of limitation, the integral combustion shield is made of a thin flame resistant or non-flammable material, such as aluminum, which covers a portion of the firelog to control the combustion rate. The combustion shield may be adhered to the surface of the firelog, to a wrapper, or held between the wrapper and firelog, so that it shields a portion of the surface of the manufactured firelog to prevent uncontrolled amounts of oxygen within the air from reaching the firelog. The combustion shield may comprise a single element, or a multiplicity of elements, and may be formed from a solid material, for instance a metallic foil, or applied as a particulate or liquid layer to the exterior of a firelog, or its wrapper, during manufacture. The combustion shield may comprise a single section, multiple sections, overlayed sections and so forth without departing from the teachings of the present invention. For example a combustion shield may provide a removable shield portion overlaying a perforated shield, thereby allowing selection of the amount of burn rate restriction. Although a number of alternatives are available for the heat shield it should be appreciated that safety, cost, and convenience are primary marketing factors determining the practical range of alternatives. In addition, most users are “unsophisticated” and there is practical value in keeping it simple—such as a single type of heat shield, wherein log can be burned conventionally or for extended time.

Prior to setting the firelog ablaze, the user selects the orientation of the firelog, and thereby the position of the combustion shield according to the amount of heat, or duration desired. If the firelog is placed so that the combustion shield is above the material of the firelog, then the firelog burns relatively conventionally, as the shield drops away as the wrapper turns to ashes. If, however, the firelog is oriented with at least a portion of the combustion shield under the firelog, retained in place by the fireplace grate or other surface on which the firelog rests, then the consumption rate of the firelog is reduced for a portion of the duration of the burn. Combustion shields according to the present invention may be incorporated onto the surface of unwrapped firelog products, or integrated into the wrapper of a wrapped manufactured firelog, wherein it may further be selectively disengaged therefrom.

To facilitate the falling away of the combustion shield from the top of a burning firelog, the shield may be configured with a means for catching the air currents present within combustion and/or means for weighting a portion of the combustion shield. By way of example the shield may have portions which project, or have apertures to increase surface area to increase forces in response to air current. Also by way of example the weighting can be readily accomplished by adding a weight strip (e.g., iron, steel, aluminum, and so forth) to a small portion, such as one side, of the combustion shield. One preferred implementation is that of forming a shield from a metal foil which has one side rolled up, or folded up, to increase weight for that side. The combustion shield formed in this manner can still be readily attached to the firelog, or more preferably the firelog wrapper, such as using an adhesive. After the surrounding material of the wrapper has burned the force of the weight causes the combustion shield to fall from the log. It should be appreciated that by coupling the weight toward the back of the log configures the shield to fall away to the back of the log wherein it is out of sight. Another advantage of the weighted portion is that it assures that a lightweight combustion shield is not able to catch wind current and float up into the chimney.

Another aspect of the invention is the adaptation of wrapped firelogs so that they may be easily divided while retaining wrapper portions thereupon. It will be appreciated that the wrapping on a firelog generally contains the material which allows for easy ignition of the firelog. The division of a wrapped firelog allows the consumer to select the size and duration of the fire to be burned. It will further be appreciated that separated portions may be burned at the same time, wherein the heat produced will be increased while the overall burning times reduced. By way of example a two piece firelog having a first section and nested second section, of two different sizes, allows the firelog to be burned in four different ways: (1) whole—a conventional heat output and duration (e.g., three hours, four hours, or similar); (2) burn the large portion—heat slightly less than whole log, but much less duration (e.g., two hours, three hours, or similar); (3) burn the small portion—significantly less heat and duration (e.g., one hour, two hours, or similar); (4) burning small portion separated (i.e., in front of) from large portion—higher heat with less duration than conventional configuration. It will be appreciated that the shapes and relative sizes and surface areas can be changed for the two firelog sections to provide the desired set of characteristics.

Another aspect of a segmented firelog may be utilized on wrapped or unwrapped firelogs which incorporate one or more embedded materials to aid in the ignition of the firelog.

Another aspect of the invention is the adaptation of the shapes of the firelogs so that they may be utilized either separately or in combination with one another. By configuring the firelogs in this way the consumer may elect the size and duration of the fire they wish to burn at a particular time. They may use the separate sections for small fires of various short durations, or combine the sections in predetermined ways to burn a fire of extended duration or extended heat production.

By way of example, a large wrapped firelog is configured for receiving a smaller wrapped firelog, wherein either the smaller or larger firelog may be burned separately, or the two firelogs may be burned in the predetermined combination. It is preferable that the large and small logs be nested and attached to one another with a temporary attachment means, such as a conventional low-tack adhesive, which allows the firelogs to be separated without removing the wrappers. These “combinable firelogs” may be sold in boxes like traditional firelogs and they allow the consumer a choice as to the burn times and heat generated by the fire. It will be appreciated that this aspect of the invention may be combined with the firelog segmentation described above and the burning rate selectable shields wherein the consumer gains additional control over the burn rate of the firelogs.

An object of the invention is to provide a manufactured firelog product in which the consumer may select the duration of burn or the amount of heat generated by the firelog as it burns.

Another object of the invention is to provide a manufactured firelog product that may be manufactured with conventional equipment.

Another object of the invention is to provide a method of controlling the burn rate of a manufactured firelog product which can be manufactured inexpensively.

Another object of the invention is to provide a method of controlling the burn rate of a manufactured firelog product wherein the consumer can select a desired combustion rate prior to ignition of the manufactured firelog product.

Another object of the invention is to provide a mechanism by which consumers uncertain of the heat safety of their fireplaces may burn the manufactured firelog at the low intensity long duration setting.

Another object of the invention is to provide a method by which the amount of air reaching the surface of the manufactured firelog is controlled so as to alter the speed with which the firelog is consumed by fire.

Another object of the invention is to provide a method by which a non-abrupt transition between areas of the manufactured firelog consumed in flames and those protected by the shield.

Another object of the invention is to provide a method of segmenting manufactured firelogs so that the user may control the size and duration of the resultant fire.

Another object of the invention is to provide a method of making firelogs in various shapes and sizes that allow burning the manufactured firelogs individually or in nested combinations with one another.

Another object of the invention is to reduce the risk of flue fires and the associated liability, by incorporating a safety device into the firelog and cautioning the consumer to utilize only the slow burn rate setting if they are uncertain about the heat capacity of their fireplace.

Further objects and advantages of the invention will be brought out in the following portions of the specification, wherein the detailed description is for the purpose of fully disclosing preferred embodiments of the invention without placing limitations thereon.

The present invention includes a split-resistant hinged bun wherein the hinged portion of the bun is augmented with a compliant, but edible, material which reinforces the hinge area of the bun to prevent the hinge area from splitting whereupon the connected bun halves become separated. It will be appreciated that sliced buns and the attendant separation of bun halves, have been problematic for a number of centuries. Within the present invention it has been recognized that the use of a single media, that being the dough which rises to form the bread, to form the hinge area of the bun results in a bun hinge which is weak and unreliable. A portion of the roll in the present invention is configured with additional material over, or within, the hinge area whereby the resultant bun becomes split resistant. By way of example and not of limitation, the edible hinges may be joined to the dough of the bun prior to baking. The edible hinges may be fabricated from a variety of edible materials which are capable of retaining flexible properties after baking, such as rice paper, casing materials, alginic acid, agar-agar, carragheenin, pectin or other gellable polysaccharides, gelatin and similar edible pliable materials.

Alternatively, the hinge area may be reinforced by incorporating more compliant edible materials, or by the injection of materials, such as those mentioned previously, to reinforce the hinge. Preferably, the bun hinges span a large portion of the hinge area and prevent the material inserted within the bun, such as condiments, from leaking out of the bun when it is being consumed. In another aspect of the invention, the described method of providing bun hinges makes possible new forms of buns for use with hot dogs, sandwiches, and other purposes.

Another aspect of the invention provides new methods of slicing a bun to allow it to be opened up. It will be appreciated that cutters are traditionally utilized with planar rotating blades for making a planar slice through the baked bread of the bun to allow it to be opened. Alternative cutting shapes are described which may be cut using a modified cutter assembly, or may preferably be cut using a laser cutting head, wherein the bun material may be more easily configured in a desired cut shape. By way of example, the bun is cut with one or more removable inner portions that may be removed at the discretion of a user that desires to fill the interior of the bun with a large amount of material. Another aspect is also described wherein the laser may also be utilized for decorating the exterior surface of the baked good, such as “laser etching” an indicia, character, graphic, text, or combinations thereof through the darkened crust of the baked good.

The present invention described in this specification is amenable to embodiment in a number of alternative ways. One embodiment can be generally described as a split resistant baked bun, comprising: (a) a bread roll formed in a predetermined shape that is partially cut-through on a first side; and (b) a flexible edible material joined to a second side forming a hinge; (c) wherein the first side of the split-resistant bun may be opened without splitting the hinge on the second side. The flexible edible material is preferably joined to either an existing hinge formed by uncut bread material in the roll which was not cut through from the first side (comprising a typical bun hinge), or joined to separate halves which were cut through from the first side (no need for a conventional hinge if this is added to an unhinged bun).

The compliant edible material may be joined to the bread roll when it is in a dough form prior to baking, or after baking. In one embodiment the compliant edible material comprises at least one thin strip of edible material applied to the roll before baking and configured to not become brittle during baking. The flexible edible material may be perforated to increase flexibility and/or to reduce the amount of material necessary for creating the hinge. The compliant edible material may comprises a liquid form material that is applied to the exterior of the roll to increase the compliance and density of the bread roll subsequent to baking. The compliant edible material may also comprise at least one additional layer of dough, preferably having increased resiliency to hinge flexure, that is joined to the second edge of the dough of the bread roll prior to baking.

Although any convenient flexible edible material may be utilized, some preferred forms of material include, but are limited to: plasticized starch material, rice paper, casing materials, alginic acid, agar-agar, carragheenin, pectin, gellable polysaccharides, gelatin, and combinations thereof. It should be recognized that any edible material capable of retaining flexible properties after baking can be utilized. Although it may be used in any baked roll or bun, the invention is particularly well suited for use with hot dog buns, hamburger buns, sliced rolls, sliced baguettes, sliced bread rolls, or other breads which are cut open for being filled with food items. The hinge system of the invention may be utilized to create a single hinge or multiple hinges. For example, additional hinges of flexible edible material may be required in order to prevent seam breakage for additional cuts, such as beyond a conventional first cut, through the bread material.

An object of the invention is to provide a baked bun that resists splitting such that the contents contained therein may be reliably retained.

Another object of the invention is to provide a split bun that can be manufactured while retaining conventional texture and taste.

Another object of the invention is to provide a sliced bun that can be baked largely according to conventional methods.

Another object of the invention is to provide a split-resistant bun that can be produced for a price in accord with current non-split resistant buns.

Another object of the invention is to provide a bun that gives the consumer a choice as to how much food items, such as wiener and condiments, which are to be contained within the bun.

Another aspect of the invention is to provide a method of cutting baked goods in complex shapes.

Another object of the invention is to provide baked goods with “etched” indicias, graphics, text, and so forth that increase brand recognition, visual appeal, or other characteristics of the product.

Further objects and advantages of the invention will be brought out in the following portions of the specification, wherein the detailed description is for the purpose of fully disclosing preferred embodiments of the invention without placing limitations thereon.

The present invention includes a cork apparatus and method of corking a bottle, such as a wine bottle, so that the cork may be easily removed with either conventional cork removal devices or by means which are facilitated by the design of the cork apparatus. A high tensile-strength inorganic material that does not react with the contents of the bottle, and does not cause wicking of the bottle contents, is configured as a cork-engagement loop, basket, or similar retention structure to engage the exterior of the cork while proffering material sections extending from the corked bottle to facilitate the removal of the cork from the bottle. It will be appreciated that making the retention loop, or basket, from long-available organic materials, such as cotton, flax, and so forth would suffer from a number of drawbacks, such as wicking, interaction with the material in the bottle (typically wine), being insufficiently thin whereas the thickness of the material interferes with the cork-to-bottle neck interface, and insufficient strength to endure the forces applied when removing the cork from the bottle. The high tensile strength material may be formed as a netted matrix about the cork that preferably converges into at least two ribbons of material that extend from the corked bottle which may be grasped on one or more ways to facilitate cork removal. The ribbon extending from the corked bottle may comprise a loop which forms a handle, a loop into which a handle is engaged, or separate pieces that are collectively grasped by an external device to remove the cork. One described manner of collectively grasping the ribbons is by inserting a rod, as may be provided with the bottle, through apertures in the ribbons. A number of alternative grasping mechanisms can be employed to apply cork-removal pressure for removing the cork from the bottle. It will be appreciated that the use of currently available high-tensile strength inorganic materials, such as those which include carbon-fiber composites, aramid fibers, carbon nanotubes, Kevlar, and so forth, can be formed into a sufficiently thin and non-reactive retention loop, or basket, to not interfere with the cork/bottle interface while providing sufficient strength to endure forces applied during cork removal without breakage. The high-tensile strength material may be fabricated sufficiently thin so as not to impede the corking operation or to lead to a leakage problem.

If the present invention is fabricated in a structure such as a straight lateral loop which extends the length of the cork, without transverse netting or ribs, then the step-down formed between the loop and cork could ostensibly in some circumstances be considered a leakage risk. If such is the case in a given application, then a further aspect of the invention allows for the use of a netting structure around the cork which includes material around the circumference of the cork. In addition, the material of the cork retention member may be pressed into (with or without a preformed recess) or other means utilized to partially embed the material of the cork retention member within the periphery of the cork surface.

The use of the retaining loop, or basket, within the present invention leaves the interior of the cork unaffected, unharmed, and not prone to premature splitting and cracking while the cork remains in the bottle. In addition, since the retaining loop, or basket, does not substantially effect the structure of the cork, conventional cork-removal devices may be utilized, included cork-screw styles devices, cork removers employing tines that are interjected between the bottle and cork, and injection style compressed gas cork removers.

Therefore, it will be appreciated that this aspect of the present invention provides a cork that may be removed from a bottle with either conventional devices or through a self-contained, or substantially self-contained cork removal device.

An object of the invention is to enhance a cork, such as for a wine bottle, with a removal means that simplifies cork extraction.

Another object of the invention is to provide a simplified cork extraction method that remains compatible with the use of conventional cork removal devices.

Another object of the invention is to provide a cork extraction device that may be implemented in numerous ways utilizing a variety of materials.

Another object of the invention is to enhance a cork with one or more members that remain externally graspable subsequent to insertion of the cork within a bottle.

Another object of the invention is to enhance a cork for easy removal without substantially reducing the surface area of the cork which is made available to the bottle interior and which allows the retained liquid to “breath”.

Another object of the invention is to provide an enhanced cork removal method that may be readily manufactured at a low cost.

Further objects and advantages of the invention will be brought out in the following portions of the specification, wherein the detailed description is for the purpose of fully disclosing preferred embodiments of the invention without placing limitations thereon.

The present invention includes an apparatus and method for removing the fog from mirror surfaces exposed to steam, such as those in the bathroom of a consumer, so that grooming and cosmetic applications may be performed with a clear reflective view. One or more sheets of material is disposed for easy application on the surface of the mirror prior to exposure to the steam. When that portion of the mirror is to be used by the consumer for viewing, then the sheet of material is peeled away along with the adhered fog, whereupon the consumer is, at least temporarily, provided a clean reflective viewing surface within the mirror. By way of example, the invention may be implemented as defog peel sheets which self-stick to the mirror surface. Other embodiments describe defog sheets which are positioned on the mirror surface by other means, such as within a roll. In addition, the integration of heating elements and other options are described to enhance utility.

An object of the invention is provide a method and apparatus for quickly removing the fog from mirrors exposed to steam, such as in a bathroom.

Another object of the invention is to provide a method for defogging a mirror surface that is inexpensive to manufacture.

Another object of the invention is to provide a method for defogging a mirror surface that may be stored for ready use.

Another object of the invention is to provide a method for defogging a mirror surface that may convey advertising materials from sponsors.

Another object of the invention is to provide a method for defogging successive portions of a mirror surface as steam fogs prior sections.

Further objects and advantages of the invention will be brought out in the following portions of the specification, wherein the detailed description is for the purpose of fully disclosing preferred embodiments of the invention without placing limitations thereon.

### BRIEF DESCRIPTION OF THE DRAWINGS

The invention will be more fully understood by reference to the following drawings which are for illustrative purposes only:

FIG. 1 is a facing view of a breath equalizer preparation according to an aspect of the present invention, shown in a oval lozenge form.

FIG. 2 is a facing view of a breath equalizer preparation according to an aspect of the present invention, shown in a circular tablet form.

FIG. 3 is a facing view of a breath equalizer preparation according to an aspect of the present invention, shown in a gel-strip or gum form.

FIG. 4 is a facing view of a breath equalizer preparation according to an aspect of the present invention, shown as a liquid contained in a dropper.

FIG. 5 is a facing view of a breath equalizer preparation packaged in combination with convention breath fresheners according to an aspect of the present invention, shown as circular tablets packaged in a roll form.

FIG. 6 is a facing view of a breath equalizer preparation packaged in combination with convention breath fresheners according to an aspect of the present invention, shown as small oval lozenges packaged in a two-chambered container.

FIG. 7 is a flowchart of method steps for manufacturing breath equalizers according to an embodiment of the present invention.

FIG. 8 is a cross-section of a manufactured firelog having a wrapper with an integrated combustion shield according to an embodiment of the present invention.

FIG. 9 is a cross-section of a manufactured firelog shown lengthwise having a wrapper and a removable combustion shield according to an embodiment of the present invention, and shown with a pull cord for removal of the combustion shield.

FIG. 10 is a perspective view of an unwrapped firelog having an integral combustion shield according to an embodiment of the present invention.

FIG. 11 is a plan view of a combustion shield according to an aspect of the present invention, shown with apertures and pull-tabs.

FIG. 12 is a plan view of a combustion shield according to an aspect of the present invention, shown with weighted displacement means and scoring, or a cut line, to allow the shield sections to be removed separately either manually or in response to combustion.

FIG. 13 is a perspective view of a firelog according to an aspect of the present invention, shown configured for separation into predetermined sections.

FIG. 14 is a side view of the firelog of FIG. 13, showing notches within the firelog which facilitate separation into sections.

FIG. 15-17 are end views of combinable firelogs according to an aspect of the present invention, shown for burning separately or on a nested combination.

FIG. 18A-18B are cross section views of firelogs configured according to an aspect of the present invention with a wrapper that joins the log material along at least one seam.

FIG. 19A-19B are cross section views of firelogs configured according to an aspect of the present invention with a wrapper that joins the log material upon a seam which includes a conbustible material.

FIG. 20A-20B are cross section views of firelogs configured according to an aspect of the present invention for receiving and engaging a wrapper along at least one seam.

FIG. 21A-21B are cross section views of the firelogs of FIGS. 20A and 20B after attachment of a wrapper along a seam.

FIG. 22 is a side view of an elongated split-resistant roll, such as a hot-dog bun, according to an aspect of the present invention, shown with vertical hinge strips on the hinge side of the roll.

FIG. 23 is a side view of the elongated split-resistant roll of FIG. 22 shown from the slit side of the roll.

FIG. 24 is an end view of the split-resistant roll of FIG. 23.

FIG. 25 is a top view of a circular split-resistant roll, such as a hamburger bun, according to an embodiment of the present invention, shown with strip hinges.

FIG. 26 is a plan view of a single-piece hinge according to an aspect of the present invention, shown with perforations.

FIG. 27 is a plan view of a single-piece hinge with extended strips according to an embodiment of the present invention.

FIG. 28 is a plan view of a single-piece zig-zag shaped hinge according to an embodiment of the present invention.

FIG. 29 is a plan view of a triangular recess baked split-resistant roll according to an aspect of the present invention, shown with two exterior hinge sections.

FIG. 30 is a cross-section of a hinge according to an aspect of the present invention, shown with a reinforced hinge region.

FIG. 31 is a cross-section of a hinge according to an embodiment of the present invention, shown with a multiple-layer hinge region.

FIG. 32 is a cross-section of a roll whose interior was cut according to an aspect of the present invention.

FIG. 33 is a cross-section of a roll whose interior was cut according to an aspect of the present invention, shown to allow for removal by consumer of one or more internal roll sections.

FIG. 34 is a side view of a roll having “etched” indicias, graphics, or text, according to an aspect of the present invention.

FIG. 35 is a perspective view of a conventional cork being removed by a conventional corkscrew.

FIG. 36 is a perspective view of a loop-style cork retainer according to an embodiment of the present invention, shown with a integral handle section.

FIG. 37 is a perspective view of the loop-style cork retainer shown in FIG. 36, folded over a bottle top after the bottle has been corked.

FIG. 38 is a perspective view of a netting-style cork retainer according to an embodiment of the present invention, shown with an integral pull handle.

FIG. 39 is a perspective view of a set of ends for an aspect of a cork retainer according to an embodiment of the present invention, shown with a rod being inserted to facilitate cork removal.

FIG. 40 is a facing view of mirror defog peel sheets according to an embodiment of the present invention, showing four sheets that were applied to a mirror prior to it becoming fogged up, such as in a bathroom.

FIG. 41 is a facing view of the mirror defog peel sheets of FIG. 40, shown after removal of a central sheet which temporarily removes the fog from that portion of the mirror.

FIG. 42 is a facing view of a single defog peel sheet according to an embodiment of the present invention, showing an optional removal tab.

FIG. 43 is an edge view of the defog peel sheet of FIG. 42.

FIG. 44 is an edge view of an enhanced thickness defog peel sheet according to another embodiment of the present invention.

FIG. 45 is a facing view of three roll-up defog peel sheets according to another embodiment of the present invention, showing the first sheet having been rolled up on a steamed-up mirror to expose a clear mirror surface underneath.

FIG. 46 is a side view of a roll-up defog sheet as shown in FIG. 45, shown overlaying a section of mirror mounted to a wall.

FIG. 47 is a facing view of a pull-down defog sheet according to another embodiment of the present invention, shown with an optional heater assembly.

### DETAILED DESCRIPTION OF THE INVENTION

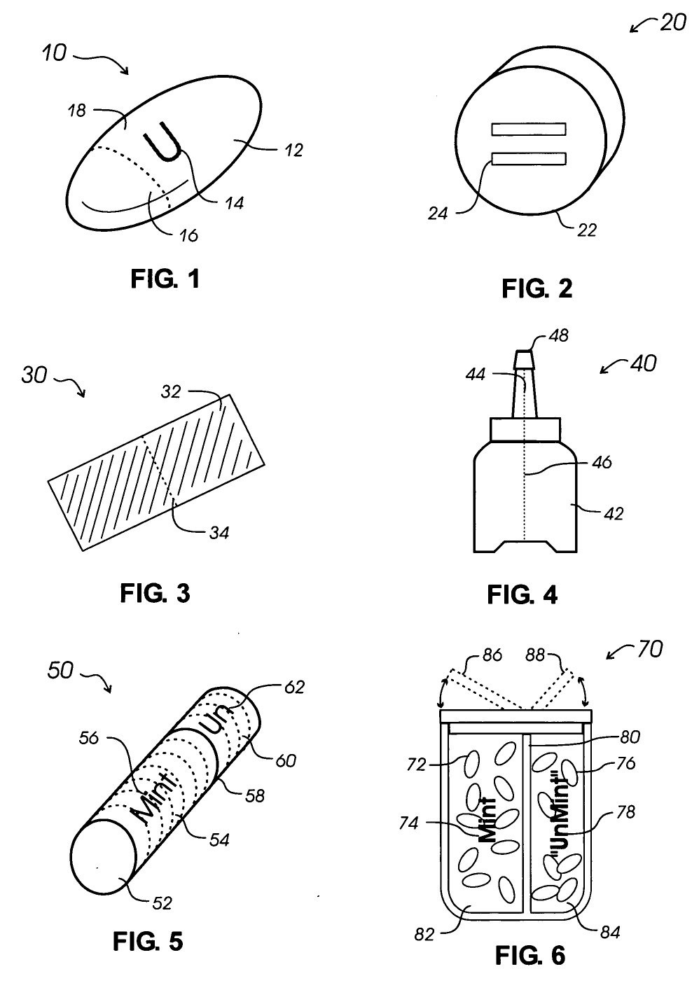

Referring more specifically to the drawings, for illustrative purposes the present invention is embodied in the apparatus generally shown in FIG. 1 through FIG. 34, and FIG. 36 through FIG. 47. It will be appreciated that the apparatus may vary as to configuration and as to details of the parts without departing from the basic concepts as disclosed herein.

The present invention depicted in FIG. 1 through FIG. 7 depicts a breath equalizer preparation according to an aspect of the present invention. Conventional breath fresheners are utilized by the person whose breath is pungent, generally as judged by another party, or parties, to reduce malodorous components. Unfortunately breath fresheners often do little more than mask pungent odors such as from garlic/onions, smoking, and so forth. The odiferous compounds, such as within garlic, are masked with a mint or other flavor, but often remain equally offensive to another party when in intimate contact.

A number of primary offending chemical substances, odiferants, have been isolated with different forms of breath halitosis. Perhaps the most common complaint, and resultant reliance on traditional breath mints, is associated with the consumption of garlic and/or onions which have a similar odiferous profile. The majority of the long-term offensive odiferous substances are within the family of thiols, which are sulfur analogs of alcohols and phenols, or sulphides. In general, the resultant halitosis is caused by less than two dozen chemical substances in the human breath, which are generally found in a person having “bad breath” in concentrations of up to 100 ppm. The smell of garlic and onions is generally in response to the presence of acrylonitrile, and allicin sulphide in concentrations of around 20 ppm. Mercaptans also play a role in the sensing of the “pungent” nature of garlic and onions. Other pungent organic compounds within both onions and garlic can be included as additional odiferants to increase the olfactory matching when sensing the breath of a person consuming the present breath unfreshener and one that has eaten the garlic or onion containing food.

The fishy smell on the breath is associated with the odiferant substance dimethylamine at concentrations around 0.05 ppm. General mouth malodors are typically the result of plaque whose primary constituents are methyl mercaptan at less than 0.001 ppm, and hydrogen sulphide at less than 0.002 ppm. It will be appreciated that the odiferous elements may be further subdivided for other foods and activities, such as smoking. It will be appreciated that the odiferants in smoking comprise various sulphides in combination with tars and nicotines, whereas an exact match of the odiferant may not be desired due to health considerations. It will further be appreciated that each person has a different level of odiferants which are generated in relation to physiological parameters, such as plaque, wherein a breath equalizer for use by the offended party may include the odiferant such as a methyl mercaptan in a very low quantity so as to reduce the offended parties sensitivity. It will be recognized that this common odiferant is generally only a byproduct of the plaque bacteria and therefore does not substantially impact actual oral hygiene.

A “breath equalizer” is therefore described herein for use by the party whose olfactory senses are being offended. It will be appreciated that by consuming a breath preparation containing even minute quantities of the odiferous elements, that the offended party will no longer be sensitive to the specific odiferous compounds. FIG. 1 through FIG. 6 depict physical embodiments of breath “equalizer” preparations as retained within a number of breath preparation carriers for oral consumption.

FIG. 1 illustrates a small oval lozenge form of tablet 10 of a base material containing odiferant 12 similar in shape to a TIC-TAC™ breath mint. As these equalizers may also be considered “unmints” the exterior of a breath “unmint” is preferably exemplified with markings 14, 16, such as the “U” 14 which stands for “unmint”, or coloring 16, that distinguish the breath equalizer from a conventional breath freshening mint. The entire lozenge/tablet or similar, may be colored 18 to differentiate them from “mints” that do not contain the “odifericant”, such as to mimic as if the eater had eaten garlic and/or onions.

FIG. 2 illustrates a circular tablet form of tableted breath equalizer 20 having a facing front surface 22 which optionally contains markings 24 indicating the equalizing nature of the mint. These unmints can be packaged within a tin, or other package, either separately by themselves, or in a compartment separated from the regular mints, or contain coloring, indicia, shape, or other means of identification which allow them to be mixed within the same container with the “regular mints” (not containing the odiferant).

In the example shown the indicia is depicted is an equal sign, although a “NO” symbol surrounding a nose symbol over a garlic symbol to indicate that this tablet eliminates a sensitivity to garlic, or onions. It will be appreciated that other symbol forms may be used to convey the concept of the breath equalizer wherein it may be distinguished from conventional breath preparations. By distinguishing the types of tablet the consumer can more readily determine which tablet is to be taken by whom.

It should also be appreciated that any of these breath medicaments can be made having two parts, a first part containing only a mint base (for the person that has eaten the garlic, or other specific offender), and a second part containing the odiferant for use by the party that has not ingested the garlic or other offender. In these examples, such as FIG. 1 or FIG. 2, the mint may be scored, or otherwise configured for separation wherein a person “shares” a mint with someone else. Preferably the halves would be color coded and/or contain indicia so that it can readily discerned which half is which.

FIG. 3 depicts the inclusion of the odiferants within a piece of gum 30 shown in stick form with a surface 32. It will be appreciated that the odiferant elements are preferably integrated within the ingredients of the gum; or are alternatively incorporated within a sugar or sugar substitute applied to its surface 32. An optional scoring 34 is shown allowing the two halves to differ in the whether they contain an odiferant and/or the type of odifericant. Preferably, if the item is scored, then at least one of the two halves is preferably marked with color, indicia, shape or other means of distinguishing what each portion contains. This figure also is representative of creating breath strips, such as formed from transparent gels, which are a popular form of breath medicament.

It should be appreciated from the above that one important aspect of the present invention is that of distributing the product having a first and second type of mint, a first containing no odiferant, and a second type containing the odiferant to “equalize” the breath between the two parties. In this way each party gets a mint—no one feels singled out. In addition, the base material of the breath preparation is preferably compatible, such as ginger, peppermint, and so forth, wherein the sensed compatibility is enhanced. The two types of breath preparation according to this aspect of the invention are distributed in combination, with mints (or mint portions) for use by the offending party and the unoffending party. In this way the package is a full solution to the particular halitosis being fought.

Alternatively, the equalizing portion may be sold separately, wherein it is made known that it may be best used in combination with another breath preparation for the other party. The equalizing breath preparation is often referred to herein, in a somewhat humerous vein, as an “unmint” portion—even though it may contain all the elements of a convention mint but include the specific odifericant for equalization purposes. Returning to the concept of packaging the unmints with mints, a manufacturer may also sell the “unmint” along with their conventional mints, and indicate the value of having both available in case a problem with onions/garlic or other pungent element could arise. A compartmentalized package may also be sold for which types of mints are sold having the odiferant and not having the odiferant; thereby allowing the consumer to purchase only the types of mint that has been used up from their package. It should be appreciated that providing a separate breath preparation containing an odiferant is taught according to the invention, while also selling in either a preferred combined form, or separately intending use in a combined form, is also fully taught within the present invention.

FIG. 4 illustrates a liquid form 40 of the invention within a container 42 having a dropper 44 to allow dispensing drops of breath equalizer. It will be appreciated that the liquid dispenser can provide a first and second half, such as divided by separating means 46, allowing dispensing of a first breath preparation without the odiferant from a first position of cap 48, and a second breath preparation containing the odiferant from a second position of cap 48. Alternatively the liquid may be dispensed from two ends of a cylindrical tube having rotatable, or removable, on the respective ends of the first and second portions.

FIG. 5 illustrates an example of packaging the dual use mints in a segmented combination roll package 50 with a printed over wrapping 52 which retains a number of disk shaped breath preparations, generally referred to as “mints” in a first portion and “unmints” in a second portion. By way of example the first portion of the package 54 contains conventional breath mints which is indicated by the information on the outer wrapping 56, such as by text and graphics. A dividing line on the package 58 is shown with a second portion of the package 60 which contain a small quantity of breath equalizer, which is preferably noted on the package 62.

FIG. 6 illustrates packaging for another embodiment of the present invention in which a combination package 70 is provided for small mints 72, 76, contained in a loose form within a container having more than one compartment. The portions of the container are marked according to purpose 74, 78, on either side of a divider 80 separating the container into a first portion 82 and a second portion 84. Access to the breath preparations is exemplified by means of hinged hatches 86, 88. It will be appreciated that a number of alternative package styles allow packaging multiple forms of breath preparations into a single package and the respective identification thereof.

The equalizing form of breath medicaments according to the present invention may be manufactured in a number of alternative ways, the following being given by way of example and not limitation. The base material of these tablets may be conventional in nature, such as utilizing sugar and sugar substitutes with added ingredients such as starches, dextrin, arabic gum, waxes, binding agents, magnesium stearate, natural and/or artificial flavors and colors, and so forth, which are compressed into a tablet form, which may be coated or uncoated. The base materials for non-solid forms of mints (e.g., liquids, gels, capsules, gums, are known in the art and need not be discussed herein).

Creating the breath equalizer according to the invention requires the addition of one or more of the odiferants associated with one or more specific foods (garlic, onions, fish) or activities (smoking, chewing) such as acrylonitrile, allicin sulphide, dimethylamine, methyl mercaptan, hydrogen sulphide, or analogous compounds, precursors, or additional organic compounds associated with the one or more particular foods, or activities, whose associated odor is to be overcome, which are included in sufficient quantity so as to result in sufficient level of malodor, to cause a degree of olfactorily insensitivity within the offended individual.

It will be appreciated that the specific composition of malodorous compounds to be included will depend on the desired target malodor, and the extent to which the breath unfreshener is to mimic the composition. Furthermore, it will be appreciated that chemical changes take place as a result of the saliva which should be considered when formulating the specific “equalizer” for use within the breath preparation. Shortly after consumption of a breath preparation according to the present invention, the olfactory senses of the individual are normalized (equalized) to the one or more particular odiferants, whereby sensitivity to this odiferant within a spouse or other party retained in close proximity is reduced or eliminated.

The benefits of using the mint with odifericant, for the non-offending party, in combination with a conventional mint, for the offending party, having the same base material the breath of both parties should be appreciated because it aids in the sensitivities of both parties becoming fully normalized to one another. It will be appreciated that conventional products attempt to mask the element that humans are sensitive to (i.e., garlic), using the mint or other elements. The use of the mint is only added to the breath resulting in breath that is still pungent but with a minty smell. In contrast the use of the breath preparation containing the odiferant (specific odiferant agent and/or precursors) for the non-offending party equalizes the aroma sensitivity of the offended party—wherein the problem is overcome. Furthermore, if both parties ingest the same mint base, then neither party is singled out for taking mint so that attention is not brought to the problem, and results in a harmonious combination that will not offend either party.

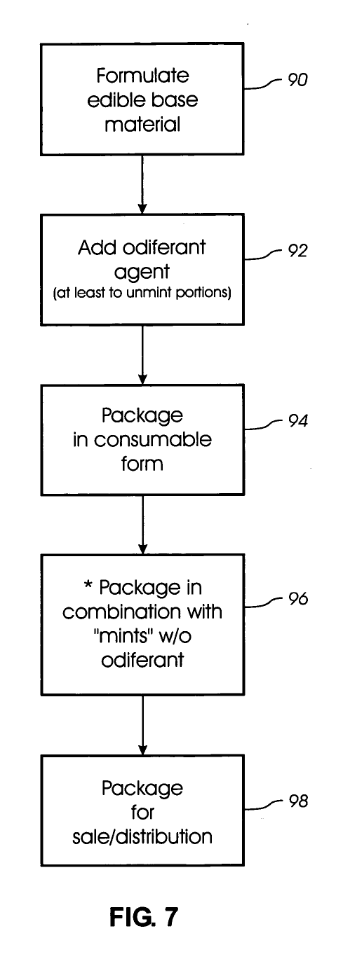

FIG. 7 depicts the general process of creating the breath equalizer according to the present invention. An edible base component, neutral or flavored (e.g., mints, ginger, cinnamon, other spices, or combinations thereof) is formulated at block 90, to which one or more of the odiferants associated with a target food, or other activity (e.g., garlic/onions, smoking, drinking) are added at block 92. The breath preparation is then formed, such as with conventional pill forming machines and so forth, as per block 94. In one preferred embodiment the breath preparation is optionally package with another breath preparation that does not contain the odifericant as represented by block 96. The breath preparation with the odifericant is then either separately packaged for sale, or in combination with a breath preparation without the odifericant, as per block 98.

Accordingly, the present invention provides a form of equalizing breath preparation that is primarily directed for consumption by the party that is offended, as opposed to the party causing the olfactory offense. The invention reduces the sensitivity of the offended party to the offending odiferant. The invention may be incorporated within conventional breath preparations, or distributed in combination thereof. It will be appreciated that the present invention describes examples of specific odiferants, and forms of packaging which can be varied by one of ordinary skill in the art without departing from the teachings of the present inventions.

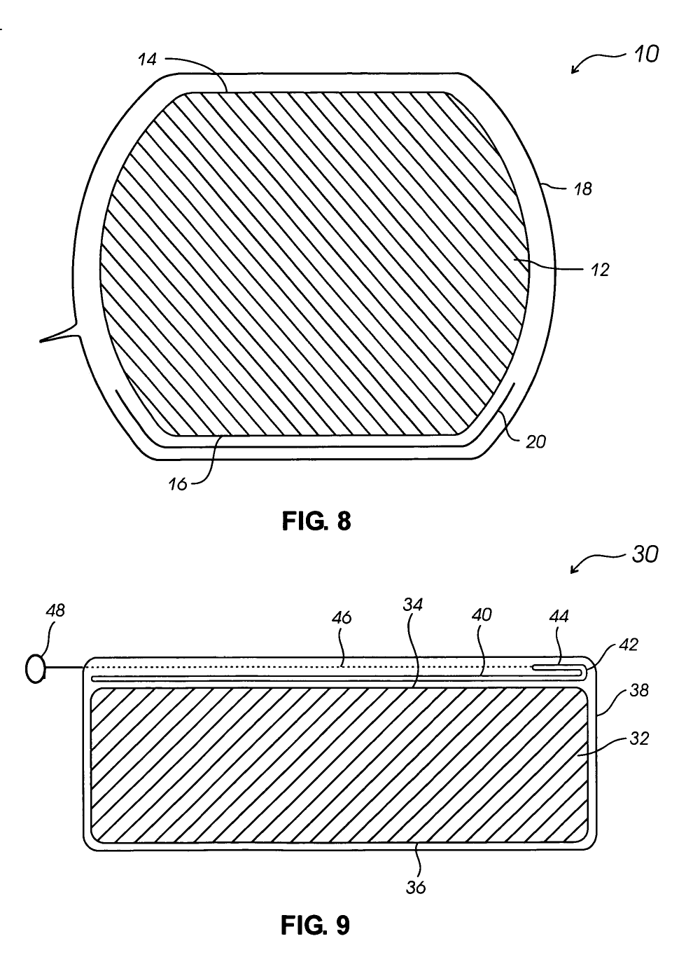

Referring first to FIG. 8 is an illustration 10 of a manufactured firelog with a wrapper having an integrated combustion shield according to the present invention. The manufactured log 12 may be of any construction, yet is typically of pressed wood particles, cardboards, and so forth which are bound together with binders such as wax. The manufactured firelog is shown by way of example with a flattened top 14 and flattened bottom 16 to facilitate positioning for lighting. It will be appreciated that the majority of manufactured firelogs have a geometric configuration wherein the log is capable of setting in a stable position in one or more orientations, such that the strip for lighting the log can be retained in a particular annular position.

To provide a clean “over-the-counter” product, to retain the moisture content, and to facilitate lighting, a wrapper 18 is generally provided. The non-flammable combustion shield 20 is shown positioned in the lower portion of the log 16 within the wrapper. The combustion shield may be fabricated of any thin non-flammable material and is preferably manufactured from a foil of aluminum. It will be appreciated that the combustion shield may alternatively be incorporated onto the log itself, retained between the log and wrapper, or integrated into the wrapper. The surface (combustion) shield may be configured to cover any portion of the log surface, generally so long as a portion of the log surface remains for initial combustion. The combustion shield may be created from a solid section of material, or it may be manufactured from sections of material. In addition, the combustion shield may be manufactured from a material which has been configured with perforations. The perforations, although they may allow limited combustion to occur through the perforations still operate to restrict the amount of combustion, and thereby reduce the overall consumption rate.

Consumption of the firelog 10 within the flame containing the combustion shield 20 can be accomplished with the shield in any orientation, however, in the embodiment shown the shield is preferably placed either at the top, or the bottom of the log while burning. It will be recognized that the exterior of the wrapper should contain instructions as per the use of the adjustable consumption firelog, and in particular the relevance of positioning the combustion shield. Burning the firelog with the shield in the downward position, as shown in FIG. 8, results in slower consumption of the firelog as less of the log surface is subject to combustion and the free flow of air. Preliminary tests with a combustion shield spanning approximately 30% of cylindrical exterior of the log surface have shown decreased consumption rates on the order of 15-20%. It is anticipated that various configurations of combustion shields will be easily capable of providing consumption rate changes of up to 35%-40%.

One advantage of using a log equipped with the combustion shield, is that it may still be burned conventionally, whereby orienting the firelog 10 with the combustion shield on the top will result in a consumption rate that is identical with a conventional firelog. As the log is ignited, the wrapper burns away and the shield falls away from on top of the log, as nothing exists to retain the shield in a fixed position. It is preferable that the combustion shield be configured to fall towards the rear of the log, so that it cannot interfere with viewing of the firelog while it is burning, however, by utilizing thin shield materials this is not required.

FIG. 9 exemplifies 30 another embodiment of the present invention wherein the combustion shield is removable prior to ignition of the firelog. The pressed firelog 32 has a top 34 and bottom 36 and a wrapping 38. Shown in this view above the log surface 34, is a combustion shield 40 which is configured for removal. To facilitate clean removal, the top portion of the shield 40 is folded over 42 with an attachment area 44 to which a pull-string 46 is connected. The pull-string exits the wrapper 38 and preferably terminates in a graspable tab 48, which may be implemented as a ring, tab, or similar easily grasped mechanism.

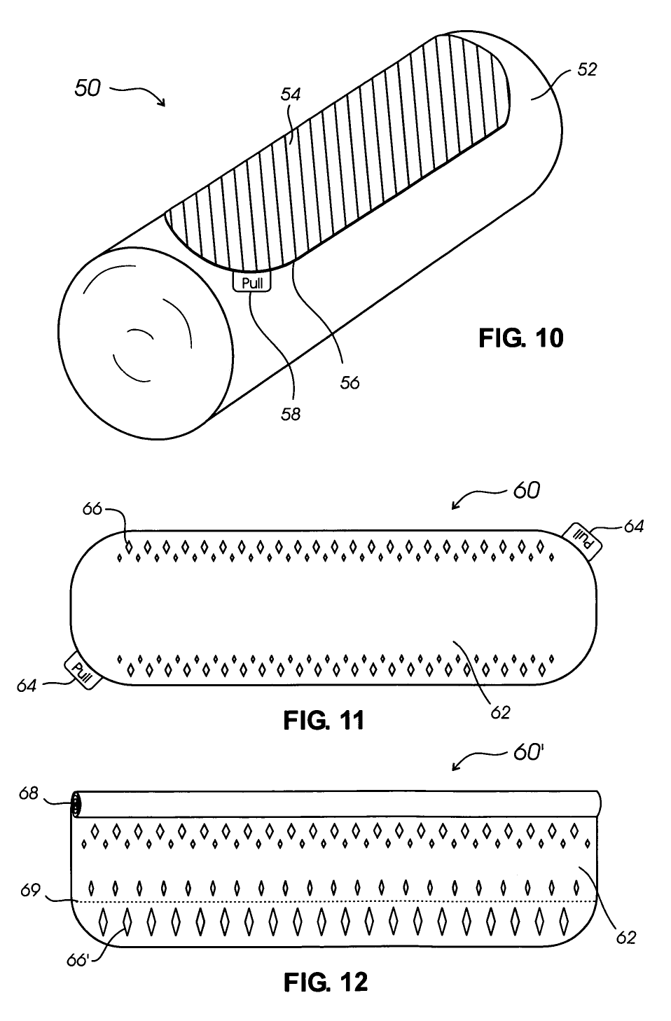

FIG. 10 exemplifies 50 an embodiment of the invention upon a firelog product that is not provided with a wrapper. The surface of the firelog 52 is configured with a combustion shield 54 attached thereupon with an outer edge 56 to which an optional tab 58 may be provided to allow for removal of the combustion shield.

FIG. 11 is a combustion shield 60 shown separated from a firelog according to an embodiment of the present invention which can be configured for application to a firelog product. The combustion shield 60 comprises a flame resistant or fireproof material 62, shown with optional tabs 64 to facilitate application and/or removal. The combustion shield is preferably formed from an inexpensive metallic material, such as a foil of aluminum, of up to approximately 30 mils thickness. It will be appreciated however, that the combustion shield need not reduce, or prevent, combustion within a particular area for the entire time period that the firelog is being burned, therefore, the combustion shield may be formed from any material that is slower burning than the agglomerated combustible material of the manufactured firelog. The shield may be attached to the surface of a firelog, or its associated wrapper so that the burning duration of the firelog may be adjusted by the consumer prior to ignition of that firelog product. The shield material 62 is shown with perforations 66 about the periphery wherein a small portion of the firelog may be partially burned to provide for a natural, non-abrupt, transition region between areas which are ignited and those which are not.

FIG. 12 is an alternate embodiment of combustion shield 60′ shown with additional optional features. First it can be seen from this embodiment that the apertures 66′ may be formed in any desired size, location and pattern. A means for biasing the combustion shield toward falling from one side of the manufactured firelog is depicted by element 68.

By way of example the means of encouraging the separation can comprise weighting a portion of the shield, such as by coupling one or more weights, or concentrating a portion of the shield material, for example depicted herein as a rolled, or folded up section of the shield (shown here in a very exaggerated roll form to be readily seen). When the combustion shield is retained over the log, therein not intended to significant alter burn characteristics, as the material surrounding the shield burn away then the weight causes shield 60′ to fall away over the back of the firelog, therein assuring its displacement from over the top of the log. It should also be appreciated that the material of the shield may be offset, in particular when situation at the top of the firelog, wherein as the wrapper burns away from the shield in the case of a wrapper mounted combustion shield, the portions of the log burn away near the edges of the shield and wind created by the rising heat and/or the use of a weight urges the shield to fall away from the firelog. It is preferred that the log be shaped so that it slopes away toward the back thereby decreasing the static friction between the log and the segments, particularly within a wrapper mounted combustion shield.

Another option depicted is that of allowing portions of the heat shield to be removed, such as if the heat shield is retained on an exterior portion of the firelog. Shield 60′ is shown optionally adapted with segmentation 69, allowing the lower portion to be removed to further control burning duration and configuration.

It will further be appreciated that the material of the combustion shield may be selected to provide numerous effects. For example, the shield may be constructed so that it disintegrates after a predetermined period of time has elapsed, wherein the log as it gets smaller is then allowed to burn sans the combustion shield. In addition, the combustion shield may be constructed of a mixture of materials, for instance whereby the edges of the material deter burning for a shorter duration that the materials chosen for the core of the combustion shield.

Using of materials with slightly different flammable volatility, firelogs may be manufactured with more controlled burning profiles. These firelogs would be preferably manufactured with a modified extrusion process wherein materials of different flammability are formed together during extrusion to form a firelog having material variability across a cross section. For example, a core of material having a higher flammability results in a firelog whose heat output can be more constant as the exterior of the firelog is burned off. It will be appreciated that within traditional firelogs the amount of heat generated is determined by the exterior surface area, wherein the amount of heat generated diminishes as the log burns toward its interior. Creating a firelog with exterior portions of different combustion material allows rotation of the firelog to control the position of different materials and thereby control selected effects, such as burn rate, direction of popping (when popping additives are added), and so forth.

In at least one embodiment the shield can be formed by adhering it to, or incorporating it within a portion of the wrapper, as previously described. Preferably the shield is glued to the inner or outer surface of the wrapper, preferably while the wrapper is still in a planar form and prior to the wrapper being wrapped and sealed about the firelog. The shield may be bonded to the wrapper with any convenient process including the use of adhesives, fasteners, stitching, interlocking reliefs or cutouts, and so forth. The shield may be created as a separate piece of material used for covering a portion of the firelog surface. The shield may be incorporated within the portions of the material of the wrapper, or a fire-proofing/fire-retardant material added as a layer to the wrapper of the log material itself. It will be appreciated that similar techniques are applicable to adding the burn shield directly to the firelog as for adding the burn shield to the wrapper.

The combustion shield may be attached to the wrapper, firelog, or an intermediate layer in a single piece, or may be segmented into sections for application thereupon. The consumer can thus further select the burning as the amount of space between segments determines the rate at which the peripheral segments will begin falling away to expose a larger portion of the log to the combustion process.

Although individual segments may be adhered to the log, wrapper, or intermediate; it may be preferable to apply a larger piece and divide into segments. For example to attach a large section of combustion shield to the surface of the log and then use a cutting device, such as a laser, cutting wheel, or mechanical cutting head to segment the material, whereupon the log may be wrapped. The direction of the combustion shield being shown on the wrapper. The small amount by which the log itself is cut into (typically 1/32″ to 1/16″) does not pose a problem.

As it may be difficult to attach and then segment a combustion shield on a wrapper, the problem may be handled in a number of ways. An additive form of shield may be created by applying one or more fire retardant layers to the wrapper (interior or exterior) as layers within a printing process, such as screen printing or similar. The shield is thus readily printed into any desired number of segments.

In a preferred embodiment the wrappers are manufactured to already include a heat shield, such as adhering them to the wrapper as it traverses a set of rollers for printing the exterior graphics. One produced, the wrapper is utilized in the log manufacture process in the same manner as the current wrappers, although it is preferred that the printing on the wrapper denote the inclusion of the combustion shield and its use. It will be appreciated that a number of fire retardant materials exist which may be applied to the wrapper (or firelog surface) in layers. As fire retardant materials are known to those of skill in the art, further details are not included herein.

A machine may be configured for applying rows of combustion shield segments to the log wrappers at the proper spacing. The segments are preferably layers with adhesive, or it is applied to the wrapper itself, just prior to application. The combustion shield may be added to a backing and then cut, or cut for adherence to a backing, whereupon the segments and backing are applied after which the backing may be removed, or remain on the log or wrapper if it is flammable. The above processes may be utilized with full coverage and/or partial coverage combustion shields (such as having a pattern of apertures to allow slight burning at portions of the shield). The combustion shield, full or segmented may be of any desired size and pattern of segmentation and may alternatively be applied to a log wrapper, or intermediary layer, without departing from the teachings of the present invention. It should also be appreciated that combination of the above techniques may be utilized without departing from the teachings of the present invention, for example the application of a removable solid combustion shield over a smaller combustion shield, a perforated combustion shield, or otherwise a combustion shield covering a smaller area thus allowing the user to select between normal burn rate and two combustion shield controlled burn rates. Alternatively, heat shields having different area may be applied to opposing sides of the log allowing the user to select between the rate provided by either log, although this is not preferred as one combustion rate replaces the normal combustion rate without shield.

FIG. 13 depicts an embodiment 70 having a firelog 72 with a wrapper 74 that are both configured for easily being separated in sections, such as along the markings 76. FIG. 14 illustrates a side view of the firelog 72 showing notches 76 which facilitate the separation of the firelog into sections, such as exemplified by the two unequal length sections shown. The notches may be configured in a number of ways that allow the firelog to be easily broken, cut, or otherwise divided by the consumer into sections. It will be appreciated that the firelog may be manufactured with any number of notches, or other similar adaptations which allow the consumer to divide the firelog to control burn rate. It will be appreciated that any lighting strip within the firelog of FIG. 13 and FIG. 14 should similarly be configured to allow the consumer to easily separate the sections of the firelog.

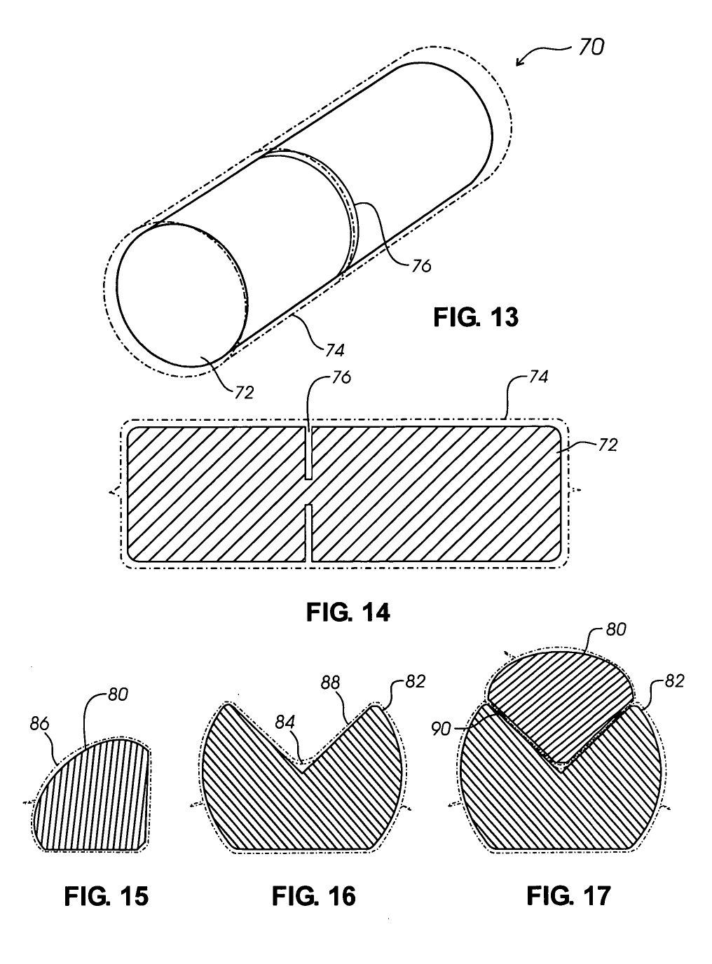

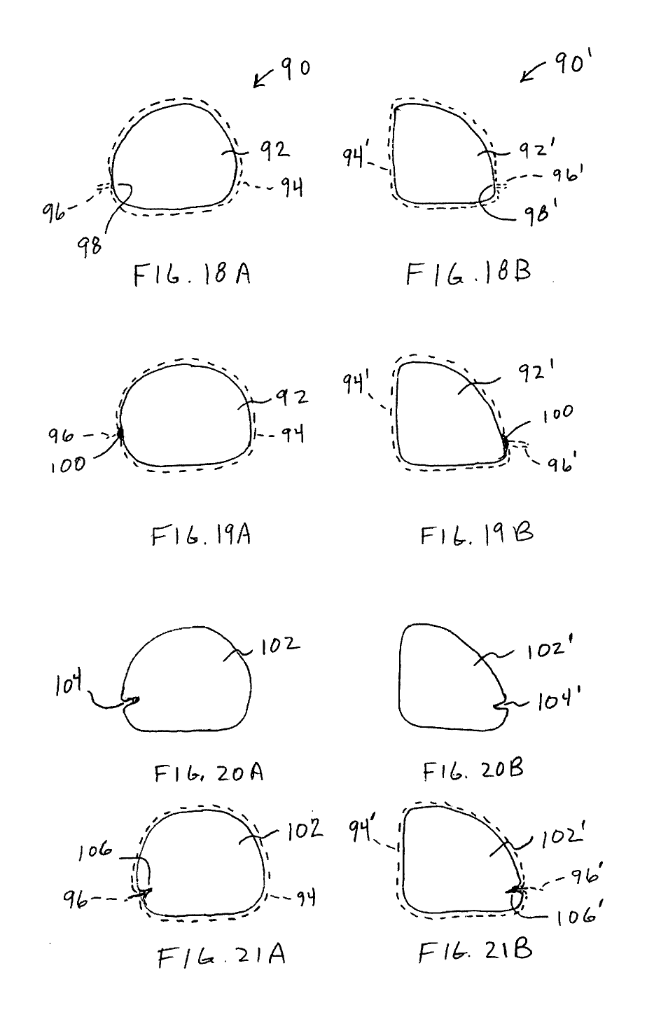

FIG. 15 through FIG. 17 illustrate another aspect of the invention which provides combinable firelogs, such as of various sizes and shapes, so that the consumer can control the duration and heat output from the fire. It will be appreciated that utilizing multiple conventional firelogs, has little impact on the duration of the fire, as the surface area of the firelogs which is exposed to the flame is independent of the number of firelogs placed in the fire. FIG. 15 depicts a small firelog 80 with a wedge-shaped cross-section, which is configured for nesting within a larger firelog 82 shown within FIG. 16 to create a combination firelog as shown in FIG. 17. The firelogs created in combinable shapes can be burned separately or combined into the larger shape of FIG. 17, depending on the desired duration and/or heat output. Preferably sudden shape changes are radiused 84, to reduce susceptibility to breakage.

Preferably, the combinable firelogs are individually wrapped to facilitate lighting and to maintain cleanliness. A wrapper is shown for the smaller section 86, and the larger portion 88, optionally including an extended portion, such as a seam that provides a lighting strip/tab extending from the wrapper. To facilitate packaging, the firelogs may also be joined, such as using a non-permanent adhesive 90, wherein the firelogs may be easily separated by the user if they want to start a fire that lasts for less duration than that provided by the combination firelog. The sections may be joined with adhesives, paper labels over the exterior joint, or other joining means which would be known to one of ordinary skill in the art, without departing from the teachings of the present invention. It will be appreciated that any number of nested firelogs, of various shapes, may be combined into the combinable firelogs of the present invention.

By way of example a two piece firelog having a first section and nested second section, of two different sizes, allows the firelog to be burned in four different ways: (1) whole—a conventional heat output and duration (e.g., three hours, four hours, or similar); (2) burn the large portion—heat slightly less than whole log, but much less duration (e.g., two hours, three hours, or similar); (3) burn the small portion—significantly less heat and duration (e.g., one hour, two hours, or similar); (4) burning small portion separated (i.e., in front of) from large portion—higher heat with less duration than conventional configuration. It will be appreciated that the shapes and relative sizes and surface areas can be changed for the two firelog sections to provide the desired set of characteristics.

By wrapping the pieces separately, and attaching the sections for easy removal the user is given a wide range of utility without the need of buying boxes of different sized logs. In a preferred embodiment, each of the sections has its own ignition strip to facilitate burning that log either separately on in combination. The nesting of the logs provides a stable configuration so that the logs are not prone to separating which changes heat output, and can be dangerous if log breaks up. The wrapping retained between the two sections provides additional support to prevent the combined sections from breaking up.

Applicant's claimed invention is structurally different and provides a number of significant advantages including containing each portion in a wrapper to prevent the mess from firelog fragments and to facilitate igniting the firelog within its wrapper. While the nested shape provides four different heat outputs (i.e. burning combination in nested mode, burning both in non-nested mode, burning the small portion, burning the large portion). Furthermore, the nested combination can remain in a stable nested configuration that maintains the desired heat output and is less subject to breaking apart.

FIG. 18A-21B illustrate by way of example embodiments another aspect of the invention in which the combination of log and wrapper is formed so as to assure ignition, speed the ignition process and promote more even burning. In these embodiments the wrapper is joined to the firelog at specific location(s) forming a specific bridge by which the combustion can begin igniting the log.

In FIG. 18A and FIG. 18B two different log cross sections 92, 92′ are shown (modified round and modified pie segment) each surrounded by a wrapper 94, 94′ which is attached to at least one portion of the firelog, such as depicted along longitudinal region 98, 98′. By way of example and not limitation 94, 94′ can be bonded with a combustible adhesive, or by using heat and pressure to sufficiently melt the surface of the log, which typically contains a wax binder, to assure bonding. It should be appreciated that wrapper 94, 94′ can be bonded to multiple locations on the surface to increase retention and to further assure that combustion is transferred to the log. It should be appreciated, however, that the wrapper at locations other than the location of the bond (including between bonding locations) should be only loosely near the surface so that the wrapper will ignite readily and burn with oxygen able to readily aid the combustion.

The portion of the wrapper to be bonded to the surface of the log can be textured, or otherwise altered, to increase adhesion, ignition, and/or appearance. For example embossing the wrapper portion at the point of attachment facilitates attachment and ignition. In another embodiment, burnable material, preferably textured, can be incorporated within the wrapper to more readily adhere to the wax and to provide additional fire starting material, to assure that the wrapper does not burn away with properly igniting the log.

In FIGS. 19A and 19B a combustion strip 100, 100′ is configured for joining wrapper 94, 94′ to the log material 92, 92′. The combustion strip may comprise any combustible material which is preferably more readily ignitable that the surface of the log. In a preferred embodiment the combustion strip is added as a pasty liquid/melt at the time the log is wrapped and the seam is pressed into the material for hardening therein.

In FIG. 20A through FIG. 21B a manufactured log and process are shown in which the wrapper is inserted into a slot or groove in the firelog to aid combustion and even burning. In FIGS. 20A and 20B log cross sections 102, 102′ are shown formed with at least one groove, or slot 104, 104′ configured for receiving the wrapper. In FIGS. 21A and 21B the wrapper 94, 94′ has been added and a portion inserted within slot 104, 104′, which is then closed up into filled slot 106, 106′ either by applying force, force and heat, the use of combustible adhesive material, or combinations thereof.

In another embodiment a piece of flammable material, such as having an “L” or “T” shaped cross-section and formed in a strip of suitable length for attachment to a manufactured firelog. The combustible fiber board may be attached to or formed integral with the manufactured firelog, leaving a portion extending from the log to join with the wrapper. The wrapper is then applied and attached adhesively, by heat and pressure, dimple adherence (i.e., surface stamped to cut through wrapper and piece). It will be appreciated that the wrapper may be joined to the strip through its seam or after the seam is formed. It is preferred, however, that the interface is configured so that if the log is handled too roughly the strip will just debond from surface of log without harming the structural integrity of the log itself, or causing the lose the integrity of the wrapper seam.

In other variations the wrapper material may be preconfiguring with a combustion material on an inner surface of the wrapper which can be adhered to the exterior of the log. The combustion aid is preferably coupled to the log in a longitudinal direction. The attachment between the wrapper and log is preferably configured that if forcefully separated, such as by hard handling or deliberate action, that (1) the integrity of the wrapper is not compromised—wherein no holes are created in the wrapper through which loose material can pass; (2) the ignition of the log is not significantly compromised, wherein the log is ignited as a conventional log with a separate, non-joined, wrapper.

In one embodiment the wrapper is attached to the log on a portion of the strip formed as a bent strip extending from the wrapper. The extra material can be formed as part of the wrapper seam, such as by creating a wide seam, or leaving extra material from one side of the wrapper extending from one side of the seam. The extra material makes the wrapper more compliant and in response to rough handling the extra section just separates from the remainder of the wrapper. To aid separation of the wrapper section, it can be segmented or otherwise configured for easy removal.

It should be appreciated that the manufactured firelog teachings above may be implemented separately or in combination with other aspects herein or that which is known in the art without departing from the present invention.

Accordingly, it will be seen that this invention of an adjustable burn rate firelog product provides a firelog, and methods for constructing a firelog, which allows the consumer to select the desired consumption rate and thereby the heat output from the firelog. It will be recognized by anyone of normal skill in the art that the invention may be implemented in a variety of ways without departing from teachings of the present invention. Embodiments have been exemplified showing configurations of firelogs and combustion shields, however, the materials and configurations are subject to wide variations without departing from the inventive teachings.

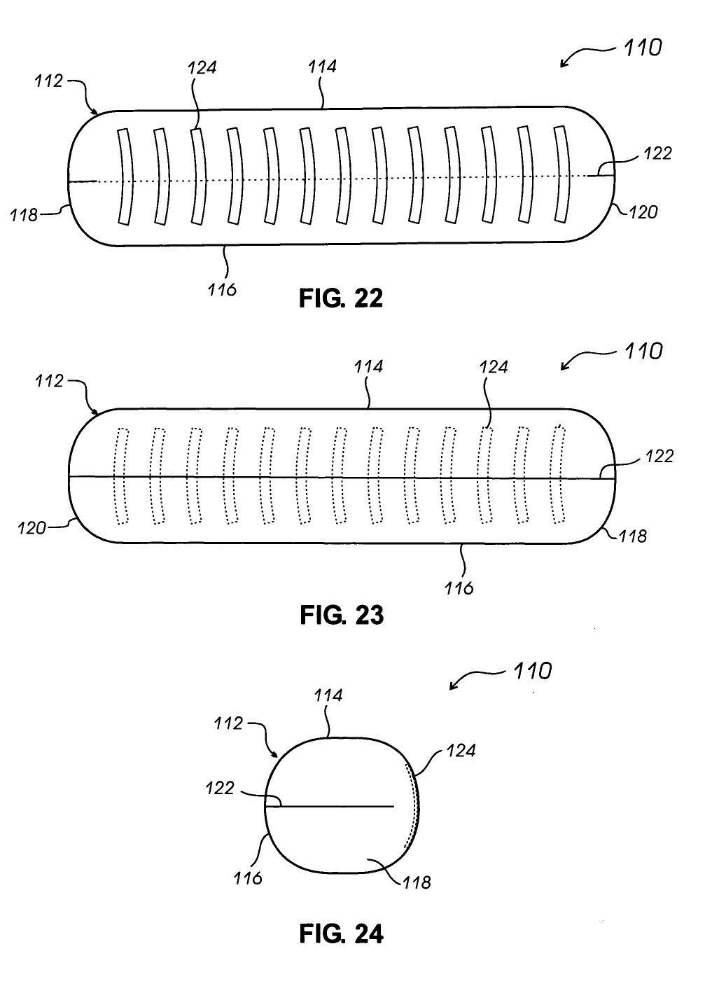

Another aspect of the invention is a split resistant bun and method of making same, example embodiments of which are depicted in FIG. 22 through FIG. 34.

FIG. 22 depicts an elongated roll 110, such as a hot dog bun, configured as a split resistant roll utilizing edible hinges according to the present invention. The bun 110 has a conventional exterior 112, top 114, bottom 116, first end 118, second end 120 and a slit 122. The bun, however, has been configured with a series of hinge elements 124 adhered near the surface of the bun. The hinges in the embodied elongated roll of the figure comprise finely perforated rectangular sections of suitably plasticized rice paper, or similar edible form of flexible material, which were adhered to, or bonded within, the dough of the bun prior to baking. The hinges retain their flexible nature after baking and remain adhered to the bun. FIG. 23 illustrates the slit side of the roll or bun, wherein the hinges are shown as hidden lines 124 rearward of the bun. After baking the roll, or bun, the slit is cut into the roll to a depth proximal the location of the hinges, whereby the hinges add strength to the remaining strip of bread that comprises a hinge upon which the halves of the bun can be rotated for the opening and closing of the bun. FIG. 24 shows the end of the hot dog bun of FIG. 22 and FIG. 23, wherein the relationship of the slit and hinges can be readily appreciated.

The hinges shown are joined with the dough of the bun prior to the baking process, such as by the application of pressure, or by utilizing an adherent. It will be appreciated that the hinges may be formed from a variety of edible materials, for instance, rice paper (or other starch based material), casings, in addition to various soy products and the like. The material of the hinge may be starch-based material to which plasticizers are added, or polymer-type materials blended. Common polymer blending materials include poly-ethylene-acrylic acid or poly-ethylene-vinyl alcohol, polyethylene, polypropylene, polystyrene, polyvinyl chloride, polyurethanes, polyesters, polyamides, polyacrylates, polyethers, polyisoprene, rubbers, and polylactides.

The hinge may comprise a region of the bun that has been adapted with a more compliant material than the remainder of the roll. The region may be made compliant by applying a liquid material, such as an egg-white mixture with a food-grade plasticizer, gelatin, or other liquids that when applied to the exterior portion of the bun will soak into the bun at the hinge area to add elasticity. A number of commercial food-grade plasticizers are available including those based on sorbitol, glycerol, sucrose, fructose, and so forth.

Materials may be injected into the hinge region of the bun to add additional material to strengthen the hinge, or to increase the compliance. Furthermore external material may be applied, either topically, or a applied as a viscous liquid which is to be built-up on the surface. Numerous forms of edible materials may comprise the build area near the hinge, such as various gellable polysaccharides, (alginic acid, agar-agar, carragheenin, pectin), and so forth. The hinge area may alternatively be “reinforced” with a material that provide another taste, such as a smoke flavor, to complement the use of the bun, or equivalent baked item. One example, would be that of a sausage casing or similar which may be applied near the hinge region of the bun to the exterior of the bun adjacent the hinge area to add additional strength. It will be appreciated that the hinge area may be reinforced with multiple layers of material which form a transitional zone, whereby the flexure stress approaching the hinge area is distributed.

Furthermore, it is not necessary that the hinges be of a thin material, as they may be constructed of thicker materials such as soya, or even dairy based products, that can provide a flavor enhancing element of the bun itself. The method of creating a split-resistant bun may be applied to a variety of baked goods.

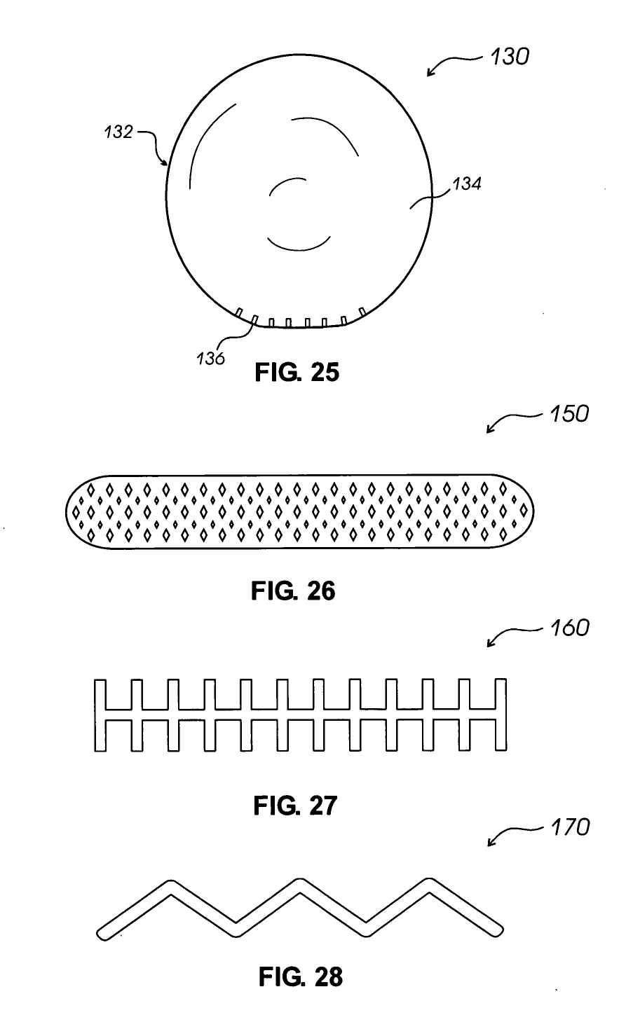

FIG. 25 shows a circular roll 130, an example of which could be a hamburger bun according to an embodiment of the present invention which is shown with perimeter hinges to provide simplifying retention of ingredients therein and split-resistance. It will be appreciated that the number of hinges may be extended wherein the roll may be stuffed similar to a pita bread.

FIG. 26 depicts an edible strip hinge according to an aspect of the present invention, wherein a stripped region of preferably perforated edible material is configured for attachment to a bun, or roll. FIG. 27 shows another form of hinge wherein small rectangular tabs extend from a central section. FIG. 28 shows another embodiment of the hinge for the present invention, wherein a zig-zag pattern of the edible hinge material is utilized. It will be appreciated that a number of shape variations of these hinges may be utilized without departing from the teachings of the present invention, wherein the embodied varieties are shown by way of example and not by way of limitation.

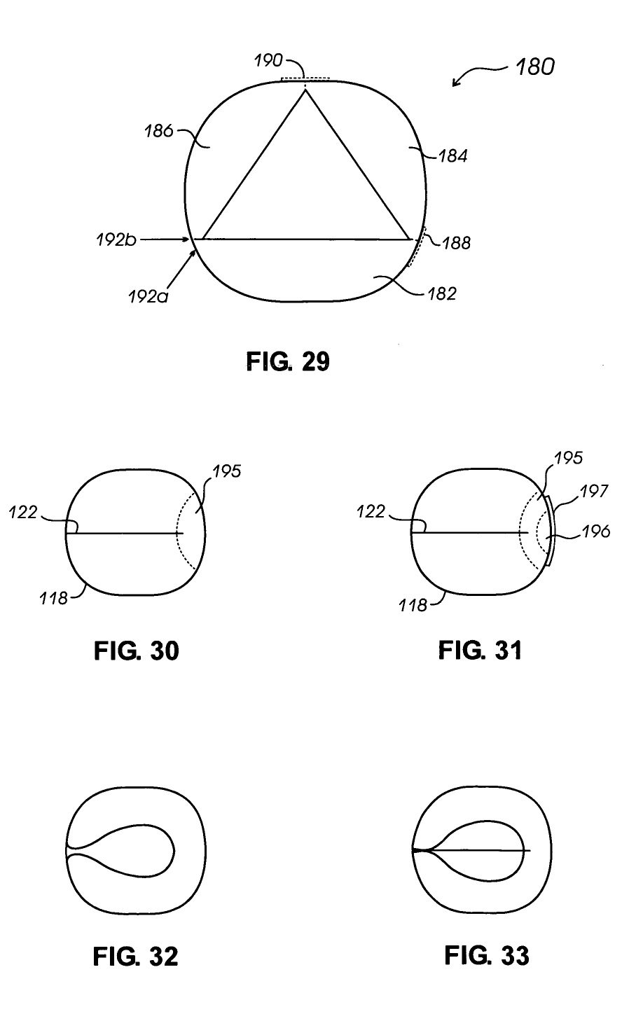

Using the described methods for reinforcing the hinges of a baked good, split-resistant buns can be created which may be easily stuffed with various fillings without splitting the hinge seam of the roll. The use of hinges facilitates the creation of new rolls for retaining foodstuffs and condiments. FIG. 29 depicts a double-hinge bun 180 that may be configured for sandwiches and sausages. A base 182 and two upper sides 184, 186 form the perimeter of the bun. Hinging the three elements 182, 184, and 186 together are two hinge sections 188, and 190. After baking, the roll is cut with at least two cuts 192, 194 wherein a large cavity center section 196 is produced which may be either removed, or left intact for the consumer to decide how to use the material. The double-hinge bun shown is capable of easily retaining more ingredients within the surrounding bread without leaking or splitting, and is ideal for large sandwiches and sausages.

FIG. 30 depicts a roll 118 whose hinge area 195 has been reinforced in a region at the terminating end of the slice which allows opening of the roll. The hinge area may be reinforced in a number of ways. A second dough material may be combined with the dough of the roll which provide a denser baked good or otherwise has more compliance than the conventional bread material. A liquid material may be applied to the exterior, or injected within the hinge area to permeate the interstices within the dough to increase density and/or compliance. The material added to the dough may include various forms of plasticizers, alginic acid, agar-agar, carragheenin, pectin, gellable polysaccharides, gelatin and so forth which can reinforce the hinge to prevent split-through when the roll is loaded with edible materials.

FIG. 31 depicts multiple layers of reinforcement 195, 196, which provide a reinforced transition region that is less abrupt than with a single region of material. An exterior layer 197 is shown attached to the exterior of the hinge area, which can provide a compliant hinge reinforcement and/or shield the area of the hinge to prevent drying of the reinforcing material of the hinge area, such as may be removed after baking.

A wide variety of materials and processes may be utilized to create the reinforcement of the hinge area of the bun. It will also be appreciated that the reinforcement may be added at any stage prior to baking or after baking. These variations will be obvious to one of ordinary skill in the art and their application does not depart from the teachings of the present invention. It is anticipated that the reinforcement material may include colorings and flavorings, such as meat flavors, condiment flavors, exotic flavors, and so forth to increase the appeal of the bun article.

The ability of a roll to retain materials and condiments may also be enhanced by creating a pocket within the interior of the roll. Conventional bun cutting devices slice a planar slot part-way through a roll but are unable to create a pocket. A second stage of cutting may be utilized wherein a router-style bit, or equivalent, opens up the interior of the roll to provide additional area for storing edibles and condiments. Furthermore, a laser can be configured as a cutting device to cut any desired shape within the interior of the roll as it will be appreciated that laser cutting techniques have progressed and have now become safe and economical for such general use.

FIG. 32 depicts a roll whose interior was cut in a tear drop pattern, such as by a laser cutting head. It can be seen that the split-cut of the bun encircles a center portion of the bun. This center section may be removed, during manufacture, or may be removed by the consumer when they want to fill the bun.

FIG. 33 depicts an interior roll cut similarly to FIG. 32, in which the traditional slit cut may be augmented with the encircling cut. It will be appreciated that this arrangement will allow the consumer to use the bun in a traditional manner with all inner bread material, or to remove one or both halves of this cutaway inner bun portion prior to filling the bun with a hot dog, or other edibles. The consumer may then select how to use the bun, as they may use it conventionally, or they may remove one or both cutaway sections prior to filling of the interior of the bun.

The bun is preferably cut using a laser cutting head that is configured with sufficient power to cut through the length of said roll. It will be appreciated that laser cutting heads may be alternatively positioned at either end of the roll being cut. Although a single cut may be made, straight or curved, the laser cutting head is preferably used to both hinge the bun and create one or more internal cutouts into which a food item may be received. To reduce discoloration, such as by charring, the laser cutting operation may be performed within a non-oxidizing gas, such as carbon dioxide, or nitrogen.

The following is a general method of cutting a baked roll into a bun. Baking of the baking of a dough roll to create a baked roll which is then aligned (one end and lengthwise axis) with at least one laser cutting head configured to emit a collimated beam of sufficient power to cut through the length of said baked roll. Whereafter the collimated beam is moved in relation to the roll according to a predetermined cutout pattern that cuts from the outside of the roll to the interior creating an opening into the interior which may be filled with food items. It will be appreciated that either the roll may be moved with a stationary beam emitting from the cutting head, and/or the beam from the cutting head may be moved in relation the roll. The pattern followed may comprise a single straight or curved line or preferably circumscribes one or more cutout areas of the interior of said roll that may be removed prior to the insertion of said food items.

It is preferable that the laser cutting head being utilized provide a power output of sufficient intensity that the roll may be cut in approximately one second or less, to reduce charring and to speed the processing of the rolls. Furthermore, it is preferred that the energy from the beam passing through the roll be recovered, such as by diffusing it onto the surface of a portion of the bakery which should be heated, or directing it through a diffusing lens into an area to be heated. For example the cutting process may be positioned near an oven, wherein the laser beam upon traversing through the length of the roll is optically diffused through a lens, or similar optical diffusing element, into an oven chamber wherein the energy is utilized for heating the oven. It will be appreciated that the laser heat may be recovered in this way for heating liquids or solids in addition to the heating of interiors such as ovens.

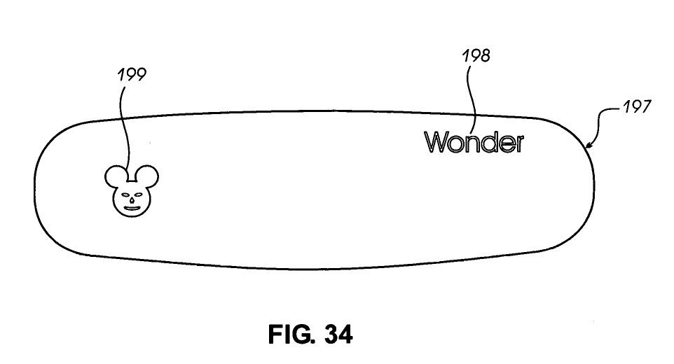

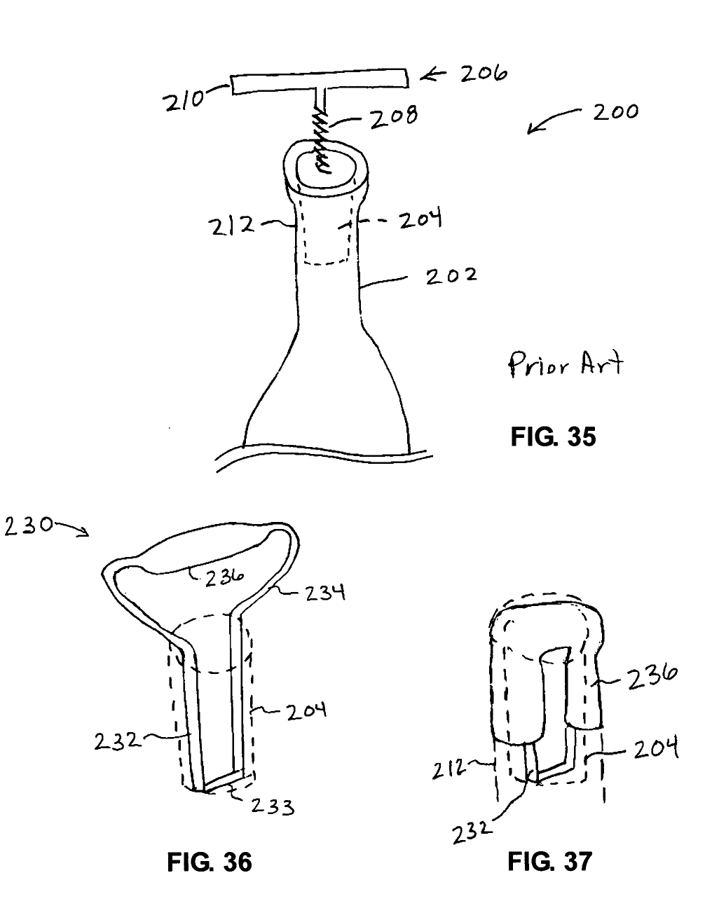

Utilizing laser cutting of baked goods provides additional capabilities as shown in FIG. 34, which depicts a roll 197 having “etched” indicias, such as text 198, and graphics 199. The power of the laser is focused at the surface of the roll, wherein it etches the dark skin of the roll to expose the lighter colors underneath. The “etching” of baked goods by a bakery can increase brand recognition, and increase sales by providing entertaining shapes, logos, and so forth.

The steps for creating text and/or graphical elements on the surface of a baked good, can be summarized as follows: baking of the item which causes the exterior crust to darkens; positioning the baked good in relation to a laser cutting head which is configured to emit a collimated beam of sufficient power to cut through the dark exterior to the lighter-colored interior; and moving the collimated beam in relation to the bakery time to follow a predetermined pattern while modulating the output of the laser cutting head according to the predetermined pattern to cut a textual or graphical element into the surface of said baked good.

It will be appreciated that the invention can be implemented in a variety of ways without departing from the teachings of the invention. In particular the hinges may comprise any of a variety of materials that are formed in a variety of shapes, while still adhering to the invention.

The present invention also includes a cork removal device (or facilitator), wherein a user need not carry a corkscrew with them, making the bottle somewhat “self-contained”.

FIG. 36 through FIG. 39 illustrate example embodiments of an on-bottle cork removal system, which incorporate a portion of the removal apparatus on the periphery of the cork. In FIG. 36 and FIG. 37 an enhanced bottle cork 230 is shown which includes a cork retention member 232 which circumscribes a portion of the exterior of the cork thus assuring that the removal forces are applied to remove the entire cork and not just its interior. In addition, by configured the removal apparatus as a thin high-tensile strength member surrounding a portion of the cork the manufactured cost of the device can be significantly reduced, such as to a few cents.

A method is described for corking a bottle that may be more easily opened than conventional corked bottles. A cork retention member 232 is exemplified in the form of a retention loop which is configured to interface between the exterior surface of cork 204, shown in phantom, and the interior of the bottle neck (not shown). A lower portion of the retention member 233 is held adjacent the lower face of the cork to direct removal pressure to the cork. The loop of cork retention member 232 is fabricated from a high-tensile strength substantially non-reactive inorganic material, such as carbon-fiber composites, aramid fibers, carbon nanotubes, Kevlar, and so forth, which may be used singly or in combinations with one another or embedded within other materials within the loop. The use of high-tensile strength material allows cork retention member 232 to be fabricated as a thin planar ribbon, preferably of a thickness less than about 20 mils and a width of approximately 1/8 inch, although designs with various sizes can be implemented from the teachings herein which provide sufficient tensile strength to withstand a pulling force of up to 20 to 40 pounds. It will be appreciated that a sufficiently thin loop, or other structural form in which a portion of the cork retention member is retained below a portion of the cork, may be inserted between the cork and bottle without disrupting the conventional bottle-to-cork seal, which is often augmented by the application of a wax material to the cylindrical exterior of the cork. Although shown as a single loop with a grasping member which comprises two pieces of material extending from the cork-to-bottle interface, it will be appreciated that the cork retention member may be implemented with any desired number of longitudinal and lateral sections of material without departing from the present invention.

To eliminate the necessity of using a specialized cork-removal device, and thereby making the cork removal essentially “self-contained” within the bottle, a grasping member is depicted as a loop of material which continues past the top of the cork as a ribbon 234 of material which joins a handle 236. The application of a sufficient pulling force to the grasping member is coupled to the cork retention member and retained cork to allow for the removal of the cork and surrounding cork retention member from the throat of a wine bottle, or equivalent. It will be appreciated that handle 236 may be adapted as a widened portion of ribbon 234, or it may include other materials to stiffen the handle area or to cushion the grip. The grasping member may thereby be configured for direct manual pulling, or manual pulling in combination with any convenient stiffener. A split plastic cover for the bottle top may be utilized as a cushion grip stiffener in combination with the handle 236. After inserting the cork with cork retention member 232 into a bottle, the extending portions of the retention member may be draped down the side or over the top of the bottle as shown in FIG. 37. A cap, such as made from lead, or plastic, may then be applied over the end of the bottle. The loop-style cork retention member of FIG. 24 is either joined to the cork prior to insertion or is retained in a given central position as the cork is inserted.

The portions of material are shown longitudinally along the sides of the cork. In some applications, the longitudinal straight-line path of cork retention member 232 along the side of the cork may be considered as a possible leakage path, such as in the case of pressurized contents, low amounts of cork wax, non-compliant cork, retention member 232 of excess thickness.

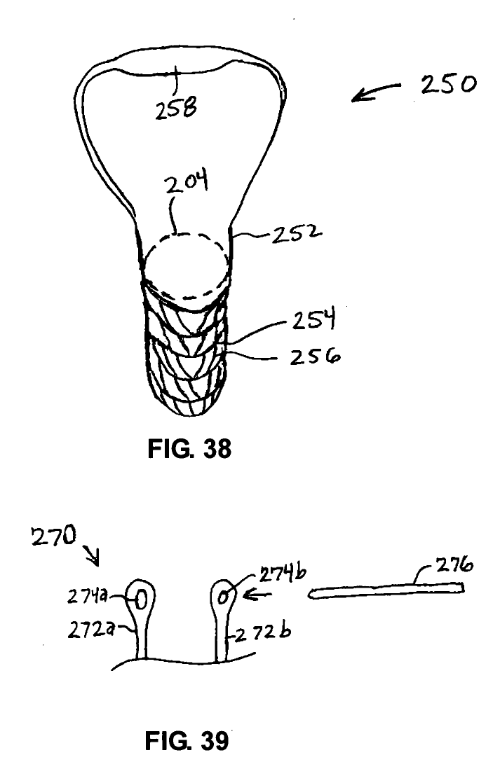

FIG. 38 illustrates an embodiment 250 of the invention which circumvent any possible leakage concerns, as the cork retention member 252 is configured with material 254 which encircles the circumference of the cork 204, while leaving a large portion of the cork surface uncovered to allow the liquid within the bottle to breath and interact with the cork material. The exemplified cork retention member 252 is exemplified as having a netting, or similar structure, comprising high tensile-strength material which retains the cork for removal and has circumferential material so that longitudinal leakage paths are cut-off. The netting, or equivalent, is shown comprising circumferential strips 254 which are joined by vertical strips 256, or strips at any angle having a vertical component. It is preferable that a portion of cork retention member 252 extend to a sufficient portion of the bottom of the cork to prevent separation from the cork in response to the application of a cork-pulling pressure. It is preferable that the cork should be retained within the cork retention member of the present invention for forces which exceed a predetermined range, based on bottle and cork material in use. The preferable force range would be between 30 pounds and 50 pounds. It will be appreciated that the core of the cork is intact within the bottle, and therefore not subject to undue cracking stress, such as may be case of retaining a threaded member within the center of the cork as provided in certain prior art devices. In addition, the combination cork and cork retention member, may be removed utilizing any conventional cork removal apparatus, as the tines of bifurcated cork removers can be inserted between the cork and bottle, and the center of the cork is unobstructed wherein corkscrews or gas pressure cork removal devices may be utilized.

The portion of the cork retention member 252 which extends outside of the corked bottle may be configured in a number of ways which facilitate the application of force to the cork retention member so that the cork may be removed. A handle 258 is shown in FIG. 38 as a convenient implementation which may be grasped by the hand, by a hand or utilized in conjunction with an elongated stiffener (not shown). It will be appreciated that the portions of the cork retention member 252 may be variously configured for grasping without departing from the teaching of the present invention.

It should be appreciated that the present invention may be purchased by a winery as cork retention members to be utilized with a selected cork size, or it may be integrated within a cork wherein the winery is able to purchase a cork that is already configured with the cork removal facilitator of the present invention.

FIG. 39 illustrates an aspect 270 of the present invention wherein the ends 272a, 272b, of the pulling strip, or mesh, are configured with apertures 274a, 274b, to facilitate applying force for removal of the cork from the bottle (not shown). By way of example, a small rod 276 is shown which may be inserted through the apertures to enable cork removal.

Accordingly, the present cork removal method and cork retention member provide a method of corking bottles such that access may be gained to the contents of the bottle, or equivalent container, utilizing a fully or partially self-contained cork removal mechanism, while still allowing for the use of conventional cork removal devices.

The present invention also includes an apparatus and method for preventing condensation on mirror surfaces, such as within a bathroom.

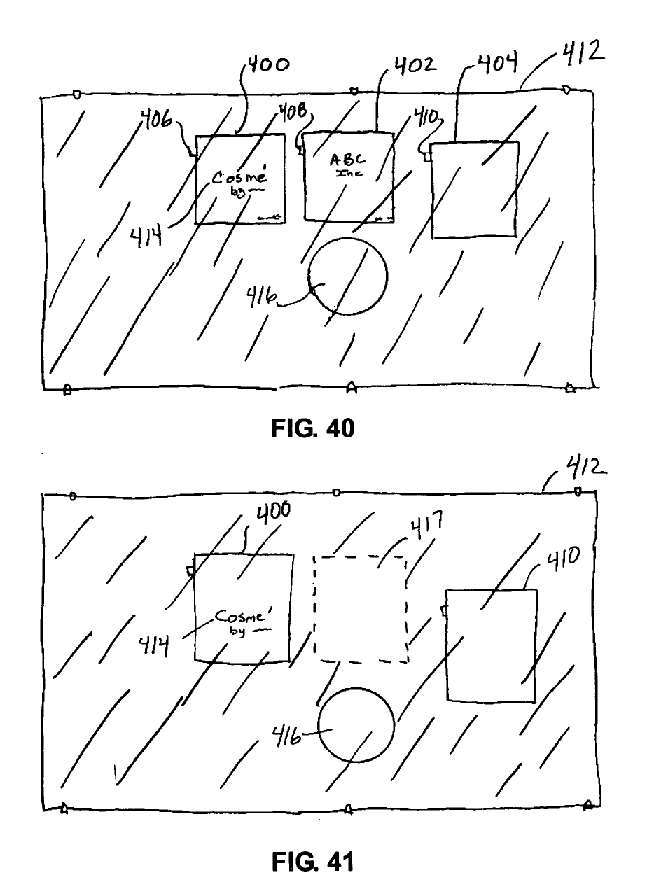

FIG. 40 through FIG. 47 illustrate example embodiments of defog peel sheets for use on mirrors by consumers, such as in a bathroom subject to shower steam, to allow removing the steam which condenses and “fogs up” the mirror surface.

FIG. 40 depicts four defog peel sheets 400, 402, 404, 416 attached to a wall-mounted mirror 412 whose surface is fogged over, such as from steam emanating from the shower. The defog peel sheets 400, 402, 404, 416, are fabricated with a substantially planar base member that is joined to an adherent layer (such as a sufficiently compliant plastic material, or reusable adhesive) on one or both planar surfaces, or portions thereof, that provides sufficient tack to allow for retention of the base member against a vertical mirror surface upon contact thereupon to prevent steam from reaching said mirror surface whereupon it may condense. It is preferable that the base material be of sufficient rigidity to simplify storage, such as by preventing the adherent layers from contacting one another. In addition the base member is preferably fabricated from a transparent material onto which indicia such as advertising may be printed. Optional removal tabs 406, 408, 410, are shown on three of the defog peel sheets to facilitate removal. The tabs 406, 408, 410, are of a material and/or orientation wherein they will not be subject to adhering to the mirror surface with the remainder of the defog peel sheet.

The defog peel sheets may be emblazoned 414 with various graphics, logos, indicias, frames depictions, and so forth. These may be preprinted, and/or applied at a later time. For example, the logo, slogan, and web site address for a cosmetics company may be imprinted during manufacture, and then additional information may be printed or otherwise applied at the cosmetics counter. It will be appreciated that an ink-jet style printer may be adapted for printing on a surface of the defog peel sheet, and the compliant (tacky) rear surface may be optional protected with a removable backing. Clear plastic labels may be applied carrying various information for the consumer, such as reorder information, phone number of cosmetics counter, name of salesperson, and so forth. In addition, information useful during the application of cosmetics may be integrated into the defog peel sheet, such as a chart of the particular consumers colors, a list of their preferred selection of cosmetics to facilitate reorder, or various other color charts and information as desired. Advertising logos may be placed on the sheets as well as other forms of trade dress, the periphery may be configured to frame any reflection therein.

When not in use, the defog peel sheets may be stored on generally unused portions of the mirror surface, such as the upper-most portion, wherein the user relocates the defog peel sheets to a necessary viewing location prior to taking a shower, or performing another activity which is expected to fog up the mirror. Since the defog peel sheets are expected to be stored on the mirror itself, their appearance should be stylish. The low manufacturing cost of the defog peel sheets makes them particularly well suited for being given away as a free gift, such as by a cosmetics company, during promotions, wherein the advertising on the defog peel sheet will be seen repeatedly by the cosmetic buying customer.

FIG. 41 illustrates fogged up mirror 412 after the user has removed defog peel sheet 402 in preparation for grooming. It will be appreciated that the fog that had condensed on the surface of sheet 402 was removed when the sheet was removed, such that the area 417 is now clear of fogging and may be used as a “portal” for viewing a specific area, such as the facial area, during grooming. It will be recognized that area 417 may begin to fog at a rate that is primarily dependent on the current level of airborne moisture and the temperature of the glass. The user may remove another of the defog peel sheets to open another “portal” when the current one becomes excessively obscured.

Each defog peel sheet is preferably of a transparent plastic material that is formed in any desired shape having sufficient surface compliancy to adhere to the glass of the mirror. It will be appreciated that a number of plastics inherently provide sufficient surface compliance and tackiness to provide adhesion to a glass surface, or may be modified to provide enough adhesion to the glass surface. For example, materials such as polyolefin resins (e.g. polyethylene and polypropylene resin), may be utilized. Adhesion strength to the glass may be increased by adding adhesives, however, the adhesive are less preferred as the use of adhesives will in general limit reusability. It is preferred that each sheet be of sufficient thickness to simplify the process of application and storage, wherein the peel sheets do not stick to themselves. Therefore, it is preferable that the material be of a thickness that exceeds approximately 30 mils. A thickness of 50 mils to 80 mils should provide sufficient thickness with most plastics to prevent wrinkling and simplify use.

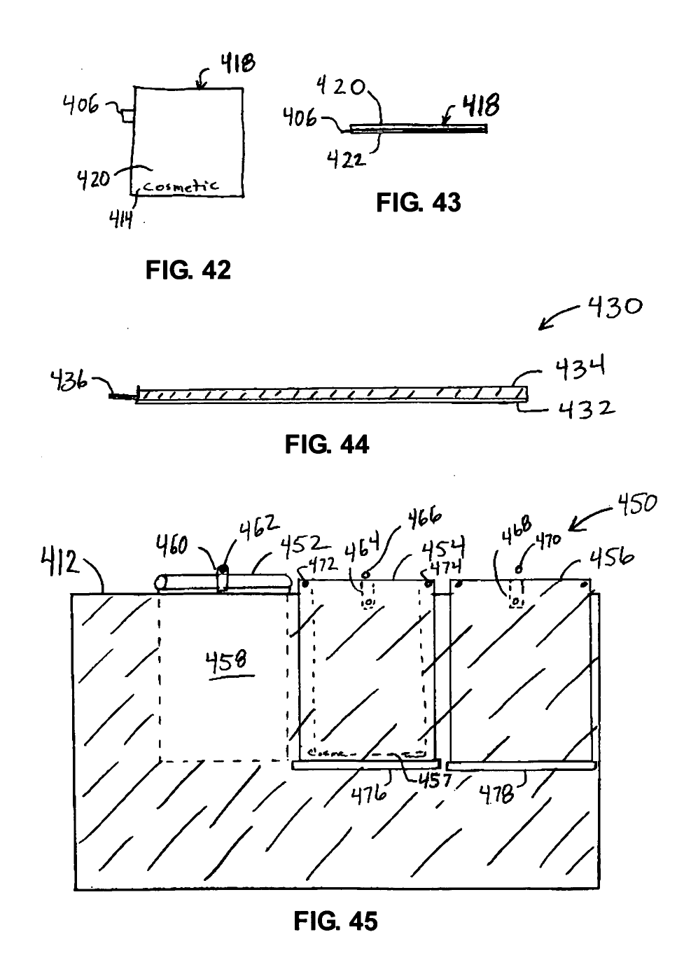

FIG. 42 and FIG. 43 illustrate another embodiment of the defog peel sheets 418 which comprise multiple layers of material. A stiff, preferably transparent, surface material 420 is joined to a plastic backing material 422 that will adhere to the surface of the mirror. Again the surface of the material may include graphics, text, or other indicia 414. To provide sufficient stiffness to facilitate application and removal the plastic which is to adhere to the mirror surface may be backed by additional material. The stiffener material may be of a less compliant plastic material, that is still preferably transparent. Providing a smooth surface on the stiffener material will increase the clarity of the view if the consumer is looking at the reflection through the stiffener. Creating transparent defog sheets, clear or shaded with color(s), is preferable as it does not block the reflective quality of the mirror, and so is considered more user friendly. In addition, it will be appreciated that the surface of the defog peel sheet may be created with a reflective coating, such as reflective Mylar, wherein the surface of the sheet mimics the surface of the underlying glass. The sheets may also be fabricated from a semi-transparent or opaque material, although this configuration fully blocks the mirror reflection and may not be as preferred an embodiment by consumers.

FIG. 44 is an enhanced thickness defog peel sheet 430 having a face 432 for adhesion to the mirror surface and a thick backing 434 with a removal tab 436. It will be appreciated that foams and other material may be incorporated within the defog peel sheet to provide rigidity. However, it should be recognized that insulating the surface of a cold mirror, and preventing it from being warmed up by the increased temperature of the steam, can lead to premature fogging during use after the defog peel sheet is removed from the mirror.

It will be appreciated that the defog peel sheets, such as shown in FIG. 44, may be configured to reduce premature fogging. Besides the addition of graphics and indicias, the material of the defog peel sheets may be configured with additives and materials to increase the effectiveness of their use. Desiccants may be incorporated within a layer of the defog sheet which is kept proximal to the mirror surface to absorb moisture. Alternatively, conventional antifogging liquids may be infused within the material of the defog peel sheet so that when a defog peel sheet is removed from the mirror the surface of the mirror has been treated with antifog compound so that fog will not accumulate on the mirror as readily as an untreated mirror. By way of example, an absorbent layer may be provided within the defog peel sheet, such as the layer 434 shown in FIG. 44, that feeds the liquid through apertures, pores, or other means of liquid conveyance to the interface with the mirror.

In addition, the material of the defog peel sheet 434 may be configured to absorb incident radiation and convert it to heat energy so as to heat up the underlying mirror. For example, the material may be made thermally conductive and supplied with a blackbody absorbing surface wherein energy radiated from the lights in the bathroom is converted to heat which is applied to the surface of the mirror. It will be appreciated that a heated mirror surface is subject to reduced condensate levels than a mirror having a lower temperature.

The defog peel sheet may also be configured to provide additional functions wherein the applied defog peel sheet may be used even when the mirror is not subject to fogging. For example the material of the defog peel sheet may be formed as a lens, such as a conventional, or a Fresnel style lens, wherein the consumer can magnify portions of the reflected image. Alignment marks and guides may be printed on the defog peel sheet to facilitate using selected cosmetics and appliances.

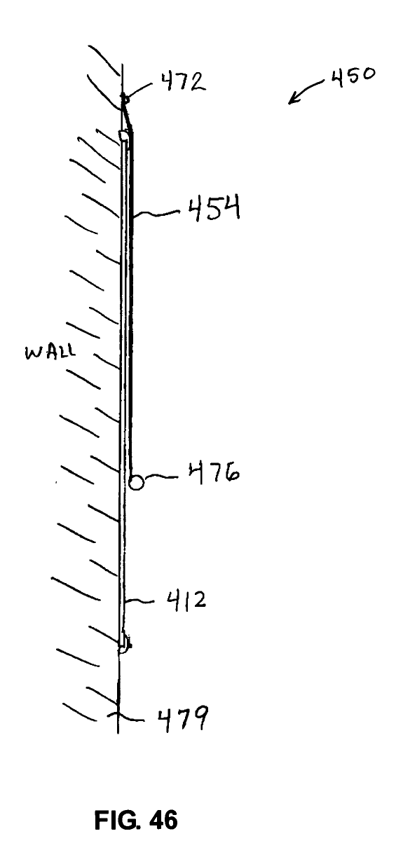

FIG. 45 and FIG. 46 are roll-up defog sheets 450 adapted for being rolled and unrolled for adherence over a mirror surface 412, shown fogged up with steam, such as from a nearby shower. FIG. 45 depicts three roll-up defog sheets 452, 454, 456, shown proximal to mirror 412. When unrolled over the mirror, one surface of the sheet is positioned against the surface of the mirror to prevent steam from condensing thereupon. It is preferable that at least edge portions 457 of the roll-up sheets are configured with an adhesive, adherent plastic edge, such as described earlier for FIG. 40, or similar retainment wherein the steam is unable to waft under exposed edges of defog sheet 454. The first roll-up defog peel sheet 452 is shown rolled up on a steamed-up mirror to expose a clear mirror surface 458 underneath. The rolled-up defog peel sheet 452 is shown rolled up above the mirror 412 being retained by a 460 and fastener 462, which can be similarly seen in the other two defog peels sheets 454, 456, with straps 464, 468, and fasteners 466, 470. The top of defog peel sheet 454 is fastened to a wall surface with fasteners 472, 474. It will be appreciated that the sheets may be fastened to the surface of the glass, such as with adhesive, or a suction-cup device, or may be fastened to other structures without departing from the teachings of the present invention. An optional weighted roll-up bars 476, 478, can be seen in the figure to facilitate quick storage away from the surface of the mirror. It will be appreciated that the implementation of the roll-up peel sheet may alternatively utilize a variety of roll-up mechanisms, for example those having latched-biasing mechanisms, which are known to those of ordinary skill in the art. FIG. 46 exemplifies how the material of the defog sheet 454 lies adjacent to the surface of mirror 412, shown attached to a wall 479.

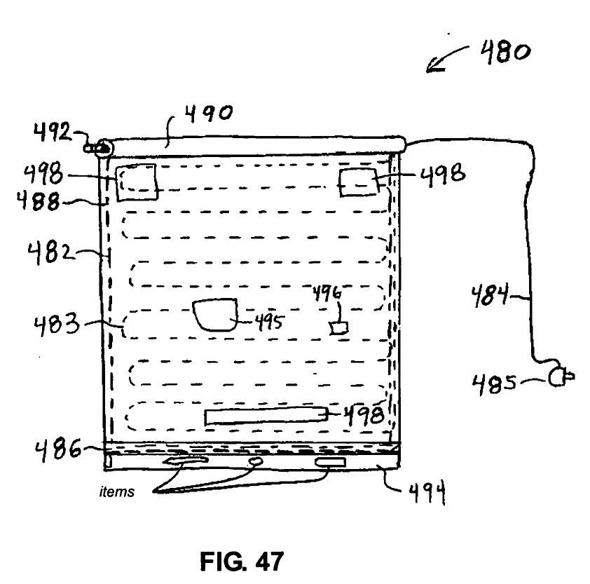

It will be appreciated that a number of variations of defog peel sheets may be implemented which operate in a similar manner to those described, such as with changes in storage or deployment, without departing from the teachings of the present invention. FIG. 47 illustrates the defog sheet 480 which is further enhanced with additional features. The defog sheet 482 has an embedded heater wire 483, which is connected through external wiring 484 to a power supply 485. Warming the surface of the glass reduces the rate at which water (fog) will condense on the surface of the mirror. It will be appreciated that mounting a roll-up defog sheet sufficiently near a light fixture can operate to heat the material of the defog peel sheet prior to application over the glass. A lower-weight bar 486 aids in keeping the sheet flat against the mirror surface. At least portions of the defog sheet, such as the lower edge and sides 488 should have a surface adherent, such as the use of a compliant plastic or an adhesive, so that the sheet is retained in close proximity with the mirror surface. The roll-up defog sheet 482 is shown attached to a spool 490 having an axial retention mount 492 with a mechanical biasing mechanism. Deploying the defog 482 sheet may be performed in similar manner to the unrolling of a window blind, wherein the defog sheet is pulled down to the desired position and then placed release or pressed against the mirror surface to assure that the sheet is held in close proximity with the glass of the mirror. The defog sheet 482 may be outfitted with storage, such as storage tray 494, pocket 495, and loop 496. A magnetic material 498 is shown attached on three locations of the defog peel sheet 480 so that magnets and lightweight items which include a sufficient quantity of magnetic material may be magnetically attached to the defog sheet. It will be appreciated that during cosmetic application a number of tools and supplies, such as cosmetic brushes, should be readily at hand. The present invention allows thin planar materials, such as pictures and so forth, to be retained by magnets which retain the planar material against the magnetic material 498. In addition, tools and other needed items may be adapted for magnetic attachment to the magnetic material 498 portion of the defog sheet. It will be appreciated that the above enhancements are generally applicable to every embodiment of the defog sheets. It will be recognized that magnetic material 498 may comprise a magnet to which magnets and ferromagnetic materials may be magnetically attached, or as a ferromagnetic material to which magnets may be magnetically attached. Inclusion of the storage compartments and locations allows the consumer to retain eyeliner brushes and other cosmetic items close to where they will be utilized. These items may be removed prior to rolling up the defog sheet or small items may be kept within the loops or fastened pockets and compartments. The surface of the sheet may be further configured to hold pictures, color charts, inspirational messages, and so forth.

Although the description above contains many specificities, these should not be construed as limiting the scope of the invention but as merely providing illustrations of some of the presently preferred embodiments of this invention. Thus the scope of this invention should be determined by the appended claims and their legal equivalents. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

1. A breath equalizer preparation, comprising:
an edible base material;
flavorings within said edible base material to enhance flavor; and
at least one odiferant agent within said edible base material which generates or simulates one or more specific causes of breath-born halitosis; and
wherein the consumption of the odiferant agent within the base material by a consumer causes a reduction in the olfactory sensitivity of said specific causes of the breath-born halitosis associated with said odiferant.

2. A breath equalizer preparation as recited in claim 1, wherein said odiferant comprises select sulphides and precursors associated with a given breath-born form of halitosis.

3. A breath equalizer preparation as recited in claim 1, wherein said odiferant is associated with the consumption of garlic, or onions.

4. A breath equalizer preparation as recited in claim 3, wherein said odiferant includes a sufficient quantity of acrylonitrile, allicin sulphide, or a combination thereof, to decrease olfactory sensitivity to garlic and onions.

5. A breath equalizer preparation as recited in claim 1, wherein said base material includes breath freshening ingredients.

6. A breath equalizer preparation as recited in claim 1, wherein said base material is selected from breath preparation carriers as selected from the group of carriers for oral consumption consisting of tablets, lozenges, chewing gums, gel strips and liquid preparations.

7. A breath equalizer preparation as recited in claim 1,
wherein said breath equalizer preparation is packaged in combination with conventional breath preparations; and
wherein portions of the package, or preparations contained therein, are marked to designate their respective use as breath freshener, or breath unfreshener.

8. A breath equalizer preparation as recited in claim 7, wherein a first consumer having a particular halitosis can take a conventional breath mint and a second consumer, not having that particular form of breath halitosis can consume the breath equalizer preparation for that particular form of halitosis which will lower their sensitivity to that particular form of halitosis present in the breath of the first consumer.

9. A method of creating a breath equalizer preparation which reduces the sensitivity of the person consuming said preparation to specific elements of breath-born halitosis, comprising:
formulating an edible base material;
adding specific odiferants to said base material which are associated with specific forms of breath-born halitosis; and
packaging said base material into a consumable form.

10. A method as recited in claim 9, further comprising packaging said breath equalizer preparation in combination with conventional breath fresheners.

11. A method as recited in claim 9, further comprising marking the breath equalizer preparation so that it may be discerned from conventional breath preparations which are directed only at attempting to freshen the breath.

12. A split resistant baked bun, comprising:
a bread roll formed in a predetermined shape that is partially cut-through on a first side; and
a flexible edible material joined to a second side forming a hinge;
wherein said first side of said split-resistant bun may be opened without splitting said hinge on said second side.

13. A split resistant baked bun as recited in claim 12, wherein said flexible edible material is joined to either an existing hinge formed by uncut bread material in the roll which was not cut through from said first side, or joined to separate halves which were cut through from said first side.

14. A split resistant baked bun as recited in claim 12:
wherein said compliant edible material is joined to said bread roll when it is in a dough form prior to baking; and
wherein said compliant edible material comprises at least one thin strip of edible material configured to not become brittle during baking.

15. A split resistant baked bun as recited in claim 12, wherein said flexible edible material is perforated.

16. A split resistant baked bun as recited in claim 12, wherein said compliant edible material comprises a liquid form material that is applied to the exterior of said roll to increase the compliance and density of said bread roll subsequent to baking.

17. A split resistant baked bun as recited in claim 12, wherein said compliant edible material comprises at least one additional layer of dough, having increased resiliency to hinge flexure, that is joined to the second edge of the dough of said bread roll prior to baking.

18. A split resistant baked bun as recited in claim 12, wherein said edible material is selected from compliant edible materials capable of retaining flexible properties after baking which consist of plasticized starch material, rice paper, casing materials, alginic acid, agar-agar, carragheenin, pectin, gellable polysaccharides, gelatin, and combinations thereof.

19. A split resistant baked bun as recited in claim 12, wherein said baked bun is comprises a hot dog bun, a hamburger bun, a roll, bread loaf, or breads which are cut open for being filled with food items.

20. A split resistant baked bun as recited in claim 12, wherein said baked bun further comprising additional hinges of flexible edible material in order to prevent seam breakage for additional cuts, beyond said first cut, through said bread material.
